(19) United States
(12) Patent Application Publication
(10) Pub. No.: US 2025/0163021 A1
(43) Pub. Date: May 22, 2025

(54) 2-METHYL-4-PHENYLPIPERIDIN-4-OL DERIVATIVES AS INHIBITORS OF APOL1 AND METHODS OF USING SAME

(71) Applicant: Vertex Pharmaceuticals Incorporated, Boston, MA (US)

(72) Inventors: Timothy J. SENTER, Arlington, MA (US); Leslie A. DAKIN, Framingham, MA (US); Elena DOLGIKH, Boston, MA (US); Jessica H. OLSEN, Jamaica Plain, MA (US); Akira J. SHIMIZU, Framingham, MA (US); Steven D. STONE, Quincy, MA (US)

(21) Appl. No.: 18/836,463

(22) PCT Filed: Feb. 8, 2023
(86) PCT No.: PCT/US2023/012579

§ 371 Date: Aug. 7, 2024

Related U.S. Application Data
(60) Provisional application No. 63/307,933, filed on Feb. 8, 2022.

(51) Int. Cl. C07D 401/06; A61K 31/454; C07D 409/14

(52) U.S. Cl. CPC C07D 401/06; A61K 31/454; C07D 409/14

(57) ABSTRACT

The disclosure provides at least one compound, tautomer, deuterated derivative, or pharmaceutically acceptable salt chosen from compounds of Formula I, tautomers thereof, deuterated derivatives of those compounds or tautomers, and pharmaceutically acceptable salts of any of the foregoing, compositions comprising the same, and methods of using the same, including uses in treating APOL1-mediated diseases, including pancreatic cancer, focal segmental glomerulosclerosis (FSGS), and/or non-diabetic kidney disease (NDKD).

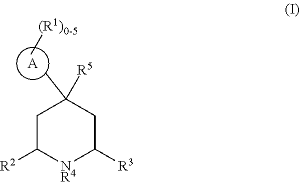

This application claims the benefit of U.S. Provisional Application No. 63/307,933, filed on Feb. 8, 2022, the contents of which are incorporated by reference in its entirety.

This disclosure provides compounds that may inhibit apolipoprotein L1 (APOL1) and methods of using those compounds to treat APOL1-mediated diseases, such as, e.g., pancreatic cancer, focal segmental glomerulosclerosis (FSGS), and/or non-diabetic kidney disease (NDKD). In some embodiments, the FSGS and/or NDKD is associated with at least one of the 2 common APOL1 genetic variants (G1: S342G:I384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV) associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed G1 and G2, which are referred to as the “APOL1 risk alleles.” G1 encodes a correlated pair of non-synonymous amino acid changes (S342G and 1384M), G2 encodes a 2 amino acid deletion (N388del:Y389del) near the C terminus of the protein, and G0 is the ancestral (low risk) allele. A distinct phenotype of NDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no or just 1 APOL1 genetic risk variant. Alternatively in AMKD, higher levels of proteinuria and accelerated loss of kidney function can also occur in patients with one risk allele. See, G. Vajgel et al., J. Rheumatol, November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing end-stage kidney disease (ESKD) and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

One aspect of the disclosure provides at least one compound, tautomer, deuterated derivative, or pharmaceutically acceptable salt chosen from compounds of Formula I, tautomers of Formula I, deuterated derivatives of those compounds or tautomers, and pharmaceutically acceptable salts of any of the foregoing, which can be employed in the treatment of diseases mediated by APOL1, such as FSGS and NDKD. For example, in some embodiments, the at least one compound is a compound of Formula I:

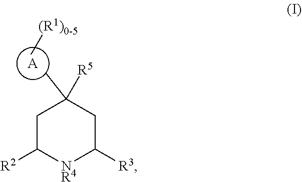

a tautomer thereof, a deuterated derivative of that compound or tautomer, or a pharmaceutically acceptable salt of any of the foregoing, wherein:
Ring A is chosen from C₆ aryl, 5- to 12-membered heterocyclyl, and 5- to 12-membered heteroaryl groups;
   R¹, for each occurrence, is independently chosen from halogen, —OH, oxo, cyano, phenyl, C₁-C₆ alkyl, C₁-C₆ alkoxy, C₃-C₆ carbocyclyl, and 4- to 6-membered heterocyclyl, wherein:
      the 4- to 6-membered heterocyclyl of R¹ comprises one heteroatom chosen from nitrogen and oxygen;
      the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
      the C₁-C₆ alkoxy of R¹ is optionally substituted with 1 to 3 groups independently chosen from —OH, cyano, and halogen groups;
      the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
      the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² and R³ are each independently chosen from hydrogen and C₁-C₄ alkyl groups;
R⁴ is chosen from C₁-C₆ alkyl, —C(═O)O(C₁-C₄ alkyl), and

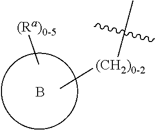

groups, wherein:
   the C₁-C₆ alkyl of R⁴ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
      the C₆ aryl and —O—(C₆ aryl) groups are each optionally substituted with 1 to 3 groups independently chosen from halogen and C₁-C₄ haloalkyl groups;
   Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
      R^{a}, for each occurrence, is independently chosen from halogen, cyano, oxo, C₁-C₈ alkyl, C₁-C₆ haloalkyl, C₂-C₈ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —C(═O)(C₁-C₄ alkylene)OR^{k}, —C(═O)R^{k}, —C(═O)(C₁-C₄ alkylene)S(═O)ₚR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k}, —NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k}, —NR^{h}C(═O)NRⁱR^{j}, —NR^{h}C(═O)(C₁-C₄ alkylene)NRⁱS(═O)ₚR^{k}, —NR^{h}S(═O)ₚR^{k}, —NR^{h}C(═O)(C₁-C₄ alkylene)S(═O)ₚR^{k}, —NR^{h}S(═O)ₚ(C₁-C₄ alkylene)C(═O)OR^{k}, —NR^{h}C(═O)[O(CH₂)_{q}]ᵣOC(═O)NR^{h}Rⁱ(CH₂)[O(CH₂)_{q}]ᵣ(C₁-C₆ alkyl) (optionally substituted by 1 to 3 R^{m} groups), —NR^{h}C(═O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
         the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)OR^{k}, —NR^{h}S(═O)ₚ(C₁-C₄ alkylene)C(═O)OR^{k}, and —NR^{h}C(═O)(C₁-C₄ alkylene)NRⁱS(═O)ₚR^{k} is optionally substituted with 1 to 3 groups independently chosen from —OH,
         the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 5- to 10-membered heterocyclyl (optionally substituted with 1 to 3 R^{m} groups), and 5- to 10-membered heteroaryl (optionally substituted with 1 to 3 R^{m} groups) groups;
         the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, oxo, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, C₆ aryl (optionally substituted with 1 to 3 R^{m} groups), and 5- to 10-membered heterocyclyl groups, wherein:
         R^{h}, Rⁱ, and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
         the C₁-C₆ alkyl of any one of R^{h}, Rⁱ, and R^{j} is optionally substituted with 1 to 3 groups independently chosen from halogen, cyano, —OH, C₁-C₄ alkoxy, —C(═O)NH(C₁-C₄ alkyl), C₃-C₆ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups;
         R^{k}, for each occurrence, is independently chosen from hydrogen, C₁-C₆ alkyl, benzyl, C₆ aryl, C₃-C₆ carbocyclyl, 5- to 10-membered heterocyclyl, and 5- to 10-membered heteroaryl groups, wherein:
         the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3 —OH groups) groups; and
         the C₃-C₆ carbocyclyl, benzyl, and C₆ aryl of any one of R^{k} are each optionally substituted with 1 to 3 groups independently chosen from halogen, cyano, oxo, —OH,
         —C(═O)NH₂, —C(═O)N(CH₃)₂, C₁-C₆ alkyl, C₁-C₆ haloalkyl,
         C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups, wherein:
         the C₁-C₄ alkyl is optionally substituted by 1 to 3 —OH groups; and the 5- to 10-membered heteroaryl and 5- to 10-membered heterocyclyl of any one of R^{k} are each optionally substituted with 1 to 3 groups independently chosen from halogen, cyano, —C(═O)CH₃, —NH₂, —OH, C₁-C₄ alkyl, C₁-C₄ haloalkyl, C₃-C₆ cycloalkyl, and C₁-C₄ alkoxy groups, wherein:
         the C₁-C₄ alkyl is optionally substituted by 1 to 3 —OH groups;
         R^{m}, for each occurrence, is independently chosen from halogen, cyano, oxo, —(CH₂).C(═O)NH₂, —NH₂, —NH(C₁-C₄ alkyl), —N(C₁-C₄ alkyl)₂, C₁-C₆ alkyl, C₁-C₆ alkoxy, —C(═O)R^{k}, —S(═O)ₚR^{k}, —OR^{k}, C₃-C₆ cycloalkyl, and 5- to 10-membered heterocyclyl groups, wherein:
         the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
R⁵ is chosen from halogen, cyano, C₁-C₆ alkyl, C₁-C₆ haloalkyl, —(CH₂).C(═O)NRⁿR^{o}, —NRⁿR^{o}, —NR^{o}C(═O)R^{P}, —NRⁿS(═O)ₚR^{P}, —(CH₂)ₙOR^{P}, —S(═O)ₚR^{P}, —S(═O)ₚNRⁿR^{o}, —OS(═O)ₚNRⁿR^{o}, and —(CH₂)ₙC(═O)OR^{p} groups, wherein:
   Rⁿ and R^{o}, for each occurrence, are each independently chosen from hydrogen and C₁-C₄ alkyl groups; and
   R^{p}, for each occurrence, is independently chosen from hydrogen, C₁-C₄ alkyl, and C₁-C₄ haloalkyl groups;
   n is an integer chosen from 0, 1, and 2;
   p, for each occurrence, is an integer independently chosen from 1 and 2; and
   q and r, for each occurrence, are each an integer independently chosen from 0, 1, 2, and 3.

In some embodiments of Formula I, at least one of R² and R³ is hydrogen and the other is chosen from C₁-C₄ alkyl groups. In these embodiments, the compound of Formula I is a compound represented by the enantiomeric structures of Formulae IIa and IIb:

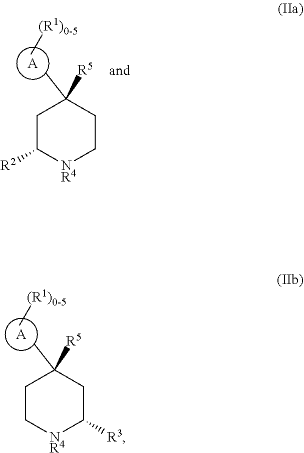

or a tautomer thereof, a deuterated derivative of those compounds and tautomers, or a pharmaceutically acceptable salt of any of the forefoing, wherein R² and R³ are chosen from C₁-C₄ alkyl groups, and Ring A, R¹, R⁴, and R⁵ are as defined above for Formula I.

In one aspect of the disclosure, the compounds of Formulae I, IIa, and IIb are chosen from Compounds 1 to 26, tautomers thereof, deuterated derivatives of those compounds and tautomers and pharmaceutically acceptable salts of any of the foregoing.

In some embodiments, the disclosure provides a pharmaceutical composition comprising at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing. In some embodiments, the pharmaceutical composition may comprise at least one compound chosen from Compounds 1 to 26, tautomers thereof, deuterated derivatives of those compounds or tautomers, and pharmaceutically acceptable salts of any of the foregoing. These compositions may further include at least one additional active pharmaceutical ingredient and/or at least one carrier.

Another aspect of the disclosure provides methods of treating an APOL1-mediated disease comprising administering to a subject in need thereof, at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing.

Another aspect of the disclosure provides methods of treating an APOL1-mediated cancer (such as, e.g., pancreatic cancer) comprising administering to a subject in need thereof, at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing.

Another aspect of the disclosure provides methods of treating APOL1-mediated kidney disease (such as, e.g., ESKD, FSGS and/or NDKD) comprising administering to a subject in need thereof, at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing.

In some embodiments, the methods of treatment include administration of at least one additional active agent to the subject in need thereof, either in the same pharmaceutical composition as the at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing, or as separate compositions. In some embodiments, the methods comprise administering at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing with at least one additional active agent, either in the same pharmaceutical composition or in a separate composition.

Also provided are methods of inhibiting APOL1, comprising administering to a subject in need thereof, at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods of inhibiting APOL1 comprise administering at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt.

### DETAILED DESCRIPTION

### Definitions

The term “APOL1,” as used herein, means apolipoprotein L1 protein and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function, and associated with 2 common APOL1 genetic variants (G1: S342G:I384M and G2: N388del:Y389del).

The term “NDKD,” as used herein, means non-diabetic kidney disease, which is characterized by severe hypertension and progressive decline in kidney function, and associated with 2 common APOL1 genetic variants (G1: S342G:I384M and G2: N388del:Y389del).

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a reference compound only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure.

Unless otherwise indicated, structures depicted herein are also meant to include all isomeric forms of the structures, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor,” as used herein, means the ratio between the isotopic abundance and the natural abundance of a specified isotope.

The term “alkyl” or “aliphatic,” as used herein, means a straight-chain (i.e., linear or unbranched) or branched, substituted or unsubstituted hydrocarbon chain that is completely saturated. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms. In some embodiments, alkyl groups contain 1 to 4 alkyl carbon atoms, in other embodiments, alkyl groups contain 1 to 3 alkyl carbon atoms, and in yet other embodiments, alkyl groups contain 1 or 2 alkyl carbon atoms. In some embodiments, alkyl groups are linear or straight-chain or unbranched. In some embodiments, alkyl groups are branched.

The terms “cycloalkyl” and “cyclic alkyl,” as used herein, refer to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The terms “carbocyclyl” or “cycloaliphatic,” as used herein, encompass the terms “cycloalkyl” or “cyclic alkyl,” and refer to a monocyclic C₃_hydrocarbon or a spirocyclic, fused, or bridged bicyclic or tricyclic C₈₋₁₄ hydrocarbon that is completely saturated, or is partially saturated as in it contains one or more units of unsaturation but is not aromatic, wherein any individual ring in said bicyclic ring system has 3 to 7 members. Bicyclic carbocyclyls include combinations of a monocyclic carbocyclic ring fused to a phenyl. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl.

The term “heteroalkyl,” or “heteroaliphatic,” as used herein, means an alkyl or aliphatic group as defined above, wherein one or two carbon atoms are independently replaced by one or more of oxygen, sulfur, nitrogen, phosphorus, or silicon.

The term “alkenyl,” as used herein, means a straight-chain (i.e., linear or unbranched) or branched hydrocarbon chain that contains one or more double bonds. In some embodiments, alkenyl groups are straight-chain. In some embodiments, alkenyl groups are branched.

The terms “heterocycle,” “heterocyclyl,” and “heterocyclic,” are used herein interchangeably to refer to non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl.

In some embodiments, the “heterocycle,” “heterocyclyl,” “heterocycloaliphatic,” or “heterocyclic” group has 3 to 14 ring members in which one or more ring members is a heteroatom independently chosen from oxygen, sulfur, nitrogen, silicon, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, silicon, and phosphorus, the quaternized form of any basic nitrogen or; a substitutable nitrogen of a heterocyclic ring, for example, N (as in 3,4-dihydro-2H-pyrrolyl), NH (as in pyrrolidinyl) or NR⁺ (as in N-substituted pyrrolidinyl)). In some embodiments, the heterocycle has one heteroatom that is a nitrogen atom. In some embodiments, the heterocycle has one heteroatom that is an oxygen atom. In some embodiments, the heterocycle has two heteroatoms that are each independently chosen from nitrogen and oxygen. In some embodiments, the heterocycle has three heteroatoms that are each independently chosen from nitrogen and oxygen. In some embodiments, the heterocyclyl is a 3- to 12-membered heterocyclyl. In some embodiments, the heterocyclyl is a 3- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. Non-limiting examples of monocyclic heterocyclyls include piperidinyl, piperazinyl, tetrahydropyranyl, azetidinyl, tetrahydrothiophenyl 1,1-dioxide, and the like.

The term “unsaturated,” as used herein, means that a moiety has one or more units or degrees of unsaturation. Unsaturation is the state in which not all of the available valence bonds in a compound are satisfied by substituents and thus the compound contains double or triple bonds.

The term “alkoxy” or “thioalkyl,” as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl,” “haloalkenyl,” and “haloalkoxy,” as used herein, mean a linear or branched alkyl, alkenyl, or alkoxy, respectively, which is substituted with one or more halogen atoms. Non-limiting examples of haloalkyl groups include —CHF₂, —CH₂F, —CF₃, —CF₂—, and perhaloalkyls, such as —CF₂CF₃. Non-limiting examples of haloalkoxy groups include —OCHF₂, —OCH₂F, —OCF₃, and —OCF₂.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

The terms “oxo” and “═O” as used herein, refer to a substituent oxygen atom connected to another atom by a double bond.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2]p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Non-limiting examples of aryl groups include phenyl (C) and naphthyl (C₁₀) rings.

The term “heteroaryl,” used alone or as part of a larger moiety as in “heteroarylalkyl” or “heteroarylalkoxy,” refers to monocyclic or spirocyclic, fused, or bridged bicyclic or tricyclic ring systems having a total of five to fourteen ring members, wherein at least one ring in the system is aromatic, wherein at least one ring in the system contains one or more heteroatoms, and wherein each ring in a bicyclic or tricyclic ring system contains 3 to 7 ring members. Bicyclic heteroaryls include the following combinations of monocyclic rings: a monocyclic heteroaryl fused to another monocyclic heteroaryl; and a monocyclic heteroaryl fused to a phenyl. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably herein and refer to an animal, including a human.

The terms “effective dose” and “effective amount” are used interchangeably herein and refer to that amount of compound that produces a desired effect for which it is administered (e.g., improvement in a symptom of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to, the following: complete or partial remission, lower risk of kidney failure (e.g., ESRD), and disease-related complications (e.g., edema, susceptibility to infections, or thrombo-embolic events). Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The at least one compound, tautomer, deuterated derivative, or pharmaceutically acceptable salt chosen from compounds of Formulae I, IIa, and IIb, tautomers thereof, deuterated derivatives of those compounds and tautomers, and pharmaceutically acceptable salts of any of the foregoing, may be administered once daily, twice daily, or three times daily, for example, for the treatment of AMKD, including FSGS and/or NDKD. In some embodiments, at least one compound chosen from Compounds 1 to 26, tautomers thereof, deuterated derivatives of those compounds and tautomers, and pharmaceutically acceptable salts of any of the foregoing may be administered once daily, twice daily, or three times daily, for example, for the treatment of AMKD, including FSGS and/or NDKD. In some embodiments, at least one compound, tautomer, deuterated derivative, or pharmaceutically acceptable salt chosen from compounds of Formulae I, IIa, and IIb, tautomers thereof, deuterated derivatives of those compounds and tautomers, and pharmaceutically acceptable salts of any of the foregoing is administered once daily. In some embodiments, at least one compound chosed from Compounds 1 to 26, tautomers thereof, deuterated derivatives of those compounds and tautomers, and pharmaceutically acceptable salts of any of the foregoing is administered once daily. In some embodiments, at least one compound, tautomer, deuterated derivative, or pharmaceutically acceptable salt chosen from compounds of Formulae I, IIa, and IIb, tautomers thereof, deuterated derivatives of those compounds and tautomers, and pharmaceutically acceptable salts of any of the foregoing is administered twice daily. In some embodiments, at least one compound chosed from Compounds 1 to 26, tautomers thereof, deuterated derivatives of those compounds and tautomers, and pharmaceutically acceptable salts of any of the foregoing is administered twice daily. In some embodiments, at least one compound, tautomer, deuterated derivative, or pharmaceutically acceptable salt chosen from compounds of Formulae I, IIa, and IIb, tautomers thereof, deuterated derivatives of those compounds and tautomers, and pharmaceutically acceptable salts of any of the foregoing is administered three times daily. In some embodiments, at least one compound chosed from Compounds 1 to 26, tautomers thereof, deuterated derivatives of those compounds and tautomers, and pharmaceutically acceptable salts of any of the foregoing is administered three times daily.

In some embodiments, 2 mg to 1500 mg or 5 mg to 1000 mg of at least one compound chosen from Formulae I, IIa, and IIb, tautomers thereof, deuterated derivatives of those compounds or tautomers, and pharmaceutically acceptable salts of any of the foregoing is administered once daily, twice daily, or three times daily. In some embodiments, 2 mg to 1500 mg or 5 mg to 1000 mg of at least one compound chosen from Compounds 1 to 26, tautomera thereof, deuterated derivative of those compounds and tautomers, and pharmaceutically acceptable salts of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound or pharmaceutically acceptable salt chosen from compounds of Formula I and pharmaceutically acceptable salts thereof” includes 1000 mg of a compound of Formula I and a concentration of a pharmaceutically acceptable salt of compounds of Formula I equivalent to 1000 mg of a compound of Formula I.

As used herein, the term “ambient conditions” means room temperature, open air condition, and uncontrolled humidity condition.

### Compounds and Compositions

In some embodiments, at least one compound chosen from Formulae I, IIa, and IIb, tautomers thereof, deuterated derivatives of those compounds and tautomers, and pharmaceutically acceptable salt of any of the foregoing may be employed in the treatment of AMKD, including FSGS and NDKD. In some embodiments, the compound of Formulae I, IIa, and IIb, may be chosen from Compounds 1 to 26, tautomers thereof, deuterated derivatives of those compounds and tautomers, and pharmaceutically acceptable salts of any of the foregoing. In some embodiments, a pharmaceutical composition comprising at least one compound chosen from Formulae I, IIa, and IIb, tautomers thereof, deuterated derivatives of those compounds and tautomers, and pharmaceutically acceptable salt of any of the foregoing, may be employed in the treatment of AMKD, including FSGS and NDKD. In some embodiments the pharmaceutical composition comprises at least one compound chosen from Compounds 1 to 26, tautomers thereof, deuterated derivatives of those compounds and tautomers, and pharmaceutically acceptable salt of any of the foregoing.

In some embodiments of Formula I:

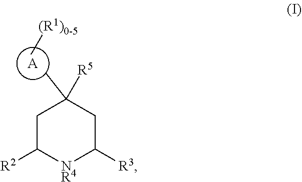

Ring A is chosen from C₆ aryl, 5- to 12-membered heterocyclyl, and 5- to 12-membered heteroaryl groups optionally substituted by 1, 2, 3, 4, or 5 R¹ groups.

In some embodiments of Formula I, Ring A is C₆ aryl. In some embodiments, Ring A is phenyl. In some embodiments, Ring A is chosen from the following:

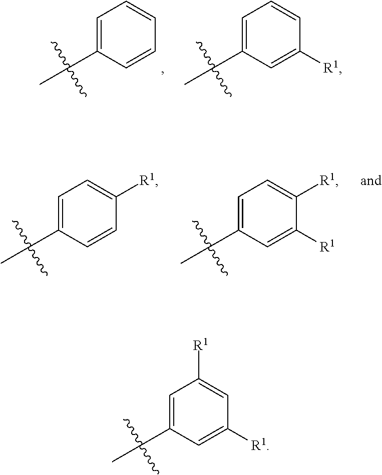

In some embodiments of Formula I, Ring A is 5- to 12-membered heterocyclyl. In some embodiments, Ring A is

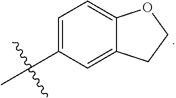 [SMILES: *c1ccc2c(c1)CCO2]

In some embodiments of Formula I (including the embodiments discussed above that define Ring A), R¹, for each occurrence, is independently chosen from halogen, —OH, oxo, cyano, phenyl, C₁-C₆ alkyl, C₁-C₆ alkoxy, C₃-C₆ carbocyclyl, and 4- to 6-membered heterocyclyl.

In some embodiments of Formula I (including the embodiments discussed above that define Ring A), the variable R¹, for each occurrence, is independently chosen from halogen. In some embodiments of Formula I (including the embodiments discussed above that define the variable Ring A), the variable R¹ is F. In some embodiments of Formula I (including the embodiments discussed above that define the variable Ring A), the variable R¹ is C₁.

In some embodiments of Formula I (including the embodiments discussed above that define Ring A), the variable R¹, for each occurrence, is independently chosen from C₁-C₆ alkyl. In some embodiments of Formula I (including the embodiments discussed above that define the variable Ring A), the variable R¹, for each occurrence, is C₁ alkyl. In some embodiments of Formula I (including the embodiments discussed above that define the variable Ring A), the variable R¹, for each occurrence, is —CH₃.

In some embodiments of Formula I (including the embodiments discussed above that define Ring A), the variable R¹, for each occurrence, is independently chosen from C₃-C₆ carbocyclyl. In some embodiments of Formula I (including the embodiments discussed above that define Ring A), the variable R¹ is C₃ carbocyclyl. In some embodiments of Formula I (including the embodiments discussed above that define Ring A), the variable R¹ is

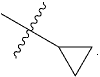 [SMILES: *C1CC1]

In some embodiments of Formula I (including the embodiments discussed above that define Ring A), the 4- to 6-membered heterocyclyl of R¹ comprises one heteroatom chosen from nitrogen and oxygen.

In some embodiments of Formula I (including the embodiments discussed above that define Ring A), the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups. In some embodiments of Formula I (including the embodiments discussed above that define Ring A), the C₁-C₆ alkyl of R¹ is optionally substituted with 1 to 3 groups independently chosen from halogen. In some embodiments of Formula I (including the embodiments discussed above that define Ring A), the C₁-C₆ alkyl of R¹ is optionally substituted with 3 halogens. In some embodiments of Formula I (including the embodiments discussed above that define Ring A), the C₁-C₆ alkyl of R¹ is optionally substituted with 3 F. In some embodiments of Formula I (including the embodiments discussed above that define Ring A), R¹ is —CF₃.

In some embodiments of Formula I (including the embodiments discussed above that define Ring A), the C₁-C₆ alkoxy of R¹ is optionally substituted with 1 to 3 groups independently chosen from —OH, cyano, and halogen groups.

In some embodiments of Formula I (including the embodiments discussed above that define Ring A), the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups.

In some embodiments of Formula I (including the embodiments discussed above that define Ring A), the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups.

In some embodiments of Formula I (including the embodiments discussed above that define Ring A and R¹), the variables R² and R³ are each independently chosen from hydrogen and C₁-C₄ alkyl groups. In some embodiments of Formula I (including the embodiments discussed above that define Ring A and R¹), the variable R² is hydrogen. In some embodiments of Formula I (including the embodiments discussed above that define Ring A and Rⁱ), the variable R² is C₁-C₄ alkyl. In some embodiments of Formula I (including the embodiments discussed above that define the variables Ring A and R¹), the variable R³ is hydrogen. In some embodiments of Formula I (including the embodiments discussed above that define Ring A and Rⁱ), the variable R³ is C₁-C₄ alkyl. In some embodiments of Formula I (including the embodiments discussed above that define the variables Ring A and Rⁱ), the variable R² is hydrogen and the variable R³ is C₁-C₄ alkyl. In some embodiments of Formula I (including the embodiments discussed above that define Ring A and Rⁱ), the variable R² is C₁-C₄ alkyl and the variable R³ is hydrogen. In some embodiments of Formula I (including the embodiments discussed above that define Ring A and R¹), the variable R² is hydrogen and the variable R³ is —CH₃. In some embodiments of Formula I (including the embodiments discussed above that define the variables Ring A and R¹), the variable R² is —CH₃ and the variable R³ is hydrogen.

In some embodiments of Formula I (including the embodiments discussed above that define Ring A, R¹, R², and R³), the variable R⁴ is chosen from C₁-C₆ alkyl, —C(═O)O(C₁-C₄ alkyl), and

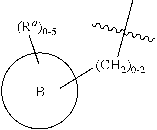

groups. In some embodiments of Formula I (including the embodiments discussed above that define Ring A, R¹, R², and R³), the variable R⁴ is chosen from

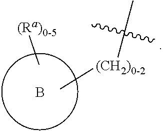

In some embodiments of Formula I (including the embodiments discussed above that define Ring A, R¹, R², and R³), the variable R⁴ is chosen from

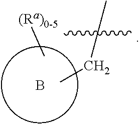

In some embodiments of Formula I (including the embodiments discussed above that define Ring A, R¹, R², and R³), the C₁-C₆ alkyl of R⁴ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups. In some embodiments, the C₆ aryl and —O—(C₆ aryl) groups of the C₁-C₆ alkyl of R⁴ are each optionally substituted with 1 to 3 groups independently chosen from halogen and C₁-C₄ haloalkyl groups.

In some embodiments of Formula I (including the embodiments discussed above that define Ring A, R¹, R², and R³), the Ring B of R⁴ is chosen from C₃-C₁₂ carbocyclyl, 3- to 12-membered heterocyclyl, C₆ and C₁₀ aryl, and 5- to 10-membered heteroaryl groups. In some embodiments of Formula I (including the embodiments discussed above that define Ring A, R¹, R², and R³), the Ring B of R⁴ is 3- to 12-membered heterocyclyl. In some embodiments of Formula I (including the embodiments discussed above that define the variables Ring A, R¹, R², and R³), Ring B of R⁴ is C₆ aryl. In some embodiments of Formula I (including the embodiments discussed above that define Ring A, R¹, R², and R³), the Ring B of R⁴ is 5- to 10-membered heteroaryl groups. In some embodiments of Formula I (including the embodiments discussed above that define Ring A, R¹, R², and R³), Ring B of R⁴ is optionally substituted with 1, 2, 3, 4, or 5 R^{a} groups. In some embodiments of Formula I (including the embodiments discussed above that define Ring A, R¹, R², and R³), Ring B of R⁴ is optionally substituted with 1, 2, or 3 R^{a} groups.

In some embodiments of Formula I (including the embodiments discussed above that define Ring A, R¹, R², and R³), Ring B of R⁴ is chosen from:

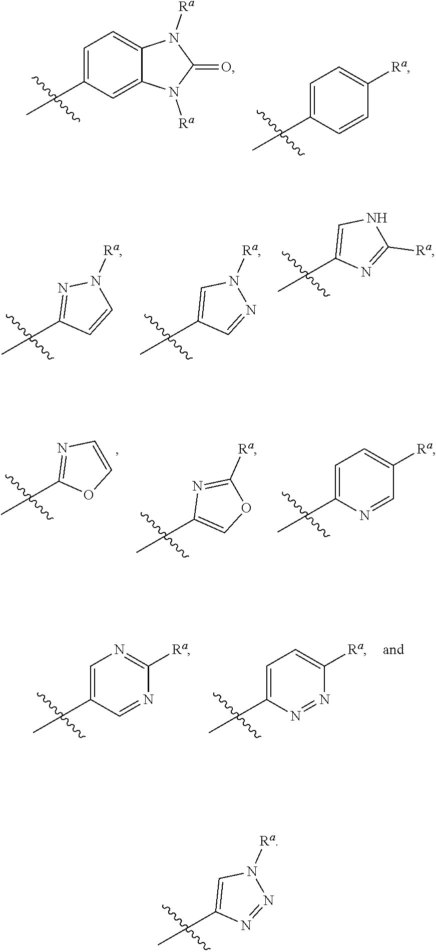

In some embodiments of Formula I (including the embodiments discussed above that define Ring A, R¹, R², and R³), Ring B of R⁴ is chosen from:

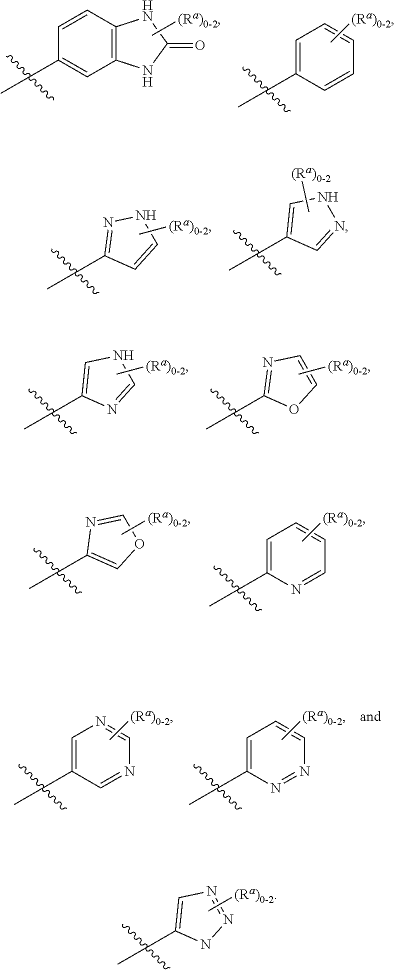

In some embodiments of Formula I (including the embodiments discussed above that define Ring A, R¹, R², and R³), the variable R^{a}, for each occurrence, is independently chosen from halogen, cyano, oxo, C₁-C₈ alkyl, C₁-C₆ haloalkyl, C₂-C₈ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —C(═O)(C₁-C₄ alkylene)OR^{k}, —C(═O)R^{k}, —C(═O)(C₁-C₄ alkylene)S(═O)ₚR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k}, —NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k}, —NR^{h}C(═O)NRⁱR^{j}, —NR^{h}C(═O)(C₁-C₄ alkylene)NRⁱS(═O)ₚR^{k}, —NR^{h}S(═O)ₚR^{k}, —NR^{h}C(═O)(C₁-C₄ alkylene)S(═O)ₚR^{k}, —NR^{h}S(═O)ₚ(C₁-C₄ alkylene)C(═O)OR^{k}, —NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3R^{m} groups), —NR^{h}C(═O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups.

In some embodiments of Formula I (including the embodiments discussed above that define Ring A, R¹, R², and R³), at least one R^{a} is oxo.

In some embodiments of Formula I (including the embodiments discussed above that define Ring A, R¹, R², and R³), the variable R^{a} is C₁-C₈ alkyl. In some embodiments of Formula I (including the embodiments discussed above that define Ring A, R¹, R², and R³), the variable R^{a} is C₁ alkyl. In some embodiments of Formula I (including the embodiments discussed above that define Ring A, R¹, R², and R³), the variable R^{a} is —CH₃. In some embodiments of Formula I (including the embodiments discussed above that define Ring A, R¹, R², and R³), the variable R^{a} is C₂ alkyl. In some embodiments of Formula I (including the embodiments discussed above that define Ring A, R¹, R², and R³), the variable R^{a} is C₃ alkyl. In some embodiments of Formula I (including the embodiments discussed above that define Ring A, R¹, R², and R³), the variable R^{a} is —CH(CH₃)₂.

In some embodiments of Formula I (including the embodiments discussed above that define Ring A, R¹, R², and R³), R^{a} is 3- to 12-membered heterocyclyl. In some embodiments of Formula I (including the embodiments discussed above that define Ring A, R¹, R², and R³), R^{a} is 5-membered heterocyclyl. In some embodiments of Formula I (including the embodiments discussed above that define Ring A, R¹, R², and R³), R^{a} is

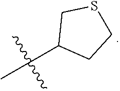 [SMILES: *C1CCSC1]

In some embodiments of Formula I (including the embodiments discussed above that define Ring A, R¹, R², and R³), R^{a} is 5- to 10-membered heteroaryl. In some embodiments of Formula I (including the embodiments discussed above that define Ring A, R¹, R², and R³), R^{a} is 6-membered heteroaryl. In some embodiments of Formula I (including the embodiments discussed above that define the variables Ring A, R¹, R², and R³), R^{a} is

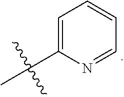 [SMILES: *c1ccccn1]

In some embodiments of Formula I (including the embodiments discussed above that define Ring A, R¹, R², and R³), R^{a} is —C(═O)NR^{h}Rⁱ. In some embodiments of Formula I (including the embodiments discussed above that define Ring A, R¹, R², and R³), the variables R^{h} and Rⁱ, for each occurrence, are each independently chosen from hydrogen and C₁-C₆ alkyl. In some embodiments of Formula I (including the embodiments discussed above that define Ring A, R¹, R², and R³), the variables R^{h} and Rⁱ are each hydrogen. In some embodiments of Formula I (including the embodiments discussed above that define Ring A, R¹, R², and R³), the variables R^{h} and Rⁱ are independently selected from C₁-C₆ alkyl. In some embodiments of Formula I (including the embodiments discussed above that define Ring A, R¹, R², and R³), one of the variables R^{h} and Rⁱ is hydrogen and the other is C₁-C₆ alkyl. In some embodiments of Formula I (including the embodiments discussed above that define Ring A, R¹, R², and R³), one of the variables R^{h} and Rⁱ is hydrogen and the other is —CH₃. In some embodiments of Formula I (including the embodiments discussed above that define Ring A, R¹, R², and R³), the variables R^{h} and Rⁱ are each —CH₃.

In some embodiments of Formula I (including the embodiments discussed above that define Ring A, R¹, R², and R³), the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)OR^{k}, —NR^{h}S(═O)ₚ(C₁-C₄ alkylene)C(═O)OR^{k}, and —NR^{h}C(═O)(C₁-C₄ alkylene)NRⁱS(═O)ₚR^{k} of R^{a} is optionally substituted with 1 to 3 groups independently chosen from —OH.

In some embodiments of Formula I (including the embodiments discussed above that define Ring A, R¹, R², and R³), the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 5- to 10-membered heterocyclyl (optionally substituted with 1 to 3 R^{m} groups), and 5- to 10-membered heteroaryl (optionally substituted with 1 to 3 R^{m} groups) groups.

In some embodiments of Formula I (including the embodiments discussed above that define Ring A, R¹, R², and R³), the C₁-C₈ alkyl, the C₁-C₆ haloalkyl, the C₁-C₆ alkoxy, and the C₂-C₈ alkenyl of R^{a} are each optionally substituted with 1 to 3 —C(═O)NR^{h}Rⁱ groups. In some embodiments of Formula I (including the embodiments discussed above that define Ring A, R¹, R², and R³), the variables R^{h} and Rⁱ, for each occurrence, are each independently chosen from hydrogen and C₁-C₆ alkyl. In some embodiments of Formula I (including the embodiments discussed above that define Ring A, R¹, R², and R³), the variables R^{h} and Rⁱ are each hydrogen. In some embodiments of Formula I (including the embodiments discussed above that define Ring A, R¹, R², and R³), the variables R^{h} and Rⁱ are independently selected from C₁-C₆ alkyl. In some embodiments of Formula I (including the embodiments discussed above that define Ring A, R¹, R², and R³), one of the variables R^{h} and Rⁱ is hydrogen and the other is C₁-C₆ alkyl. In some embodiments of Formula I (including the embodiments discussed above that define Ring A, R¹, R², and R³), one of the variables R^{h} and Rⁱ is hydrogen and the other is —CH₃. In some embodiments of Formula I (including the embodiments discussed above that define Ring A, R¹, R², and R³), the variables R^{h} and Rⁱ are each —CH₃.

In some embodiments of Formula I (including the embodiments discussed above that define Ring A, R¹, R², and R³), the C₁-C₈ alkyl, the C₁-C₆ haloalkyl, the C₁-C₆ alkoxy, and the C₂-C₈ alkenyl of R^{a} are each optionally substituted with 1 to 3 —S(═O)ₚR^{k} groups. In some embodiments of Formula I (including the embodiments discussed above that define Ring A, R¹, R², and R³), the variable p, for each occurrence, is an integer independently chosen from 1 and 2. In some embodiments of Formula I (including the embodiments discussed above that define the variables Ring A, R¹, R², and R³), the variable p is 2. In some embodiments of Formula I (including the embodiments discussed above that define Ring A, R¹, R², and R³), the variable R^{k} is chosen from C₁-C₆ alkyl. In some embodiments of Formula I (including the embodiments discussed above that define Ring A, R¹, R², and R³), the variable R^{k} is C₁ alkyl. In some embodiments of Formula I (including the embodiments discussed above that define the variables Ring A, R¹, R², and R³), the variable R^{k} is —CH₃.

In some embodiments of Formula I (including the embodiments discussed above that define the variables Ring A, R¹, R², and R³), the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, oxo, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, C₆ aryl (optionally substituted with 1 to 3 R^{m} groups), and 5- to 10-membered heterocyclyl groups. In some embodiments of Formula I (including the embodiments discussed above that define Ring A, R¹, R², and R³), the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 oxo.

In some embodiments of Formula I (including the embodiments discussed above that define the variables Ring A, R¹, R², and R³), the variables R^{h}, Rⁱ, and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3R^{m} groups), and 5- to 10-membered heterocyclyl (optionally substituted with 1 to 3 R^{m} groups) groups.

In some embodiments of Formula I (including the embodiments discussed above that define Ring A, R¹, R², and R³), the variables R^{h}, Rⁱ, and R^{j}, for each occurrence, are each independently chosen from hydrogen and C₁-C₆ alkyl. In some embodiments of Formula I (including the embodiments discussed above that define Ring A, R¹, R², and R³), the variables R^{h}, Rⁱ, and R^{j}, for each occurrence, are each independently chosen from hydrogen and C₁-C₆ alkyl. In some embodiments of Formula I (including the embodiments discussed above that define Ring A, R¹, R², and R³), the variables R^{h}, Rⁱ, and R^{j}, are each hydrogen. In some embodiments of Formula I (including the embodiments discussed above that define Ring A, R¹, R², and R³), the variables R^{h}, Rⁱ, and R^{j} are each independently selected from C₁-C₆ alkyl. In some embodiments of Formula I (including the embodiments discussed above that define Ring A, R¹, R², and R³), one of the variables R^{h}, Rⁱ, and R^{j} is hydrogen and the other two are C₁-C₆ alkyl. In some embodiments of Formula I (including the embodiments discussed above that define Ring A, R¹, R², and R³), two of the variables R^{h}, Rⁱ, and R^{j} are hydrogen and the other one is C₁-C₆ alkyl. In some embodiments of Formula I (including the embodiments discussed above that define Ring A, R¹, R², and R³), the variables R^{h}, R^{k}, and R^{j}, for each occurrence, are each independently chosen from hydrogen and —CH₃.

In some embodiments of Formula I (including the embodiments discussed above that define Ring A, R¹, R², and R³), the C₁-C₆ alkyl of any one of R^{h}, Rⁱ, and Rⁱ is optionally substituted with 1 to 3 groups independently chosen from halogen, cyano, —OH, C₁-C₄ alkoxy, —C(═O)NH(C₁-C₄ alkyl), C₃-C₆ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups.

In some embodiments of Formula I (including the embodiments discussed above that define Ring A, R¹, R², and R³), the variable R^{k}, for each occurrence, is independently chosen from hydrogen, C₁-C₆ alkyl, benzyl, C₆ aryl, C₃-C₆ carbocyclyl, 5- to 10-membered heterocyclyl, and 5- to 10-membered heteroaryl groups. In some embodiments of Formula I (including the embodiments discussed above that define Ring A, R¹, R², and R³), the variable R^{k} is C₁-C₆ alkyl. In some embodiments of Formula I (including the embodiments discussed above that define Ring A, R¹, R², and R³), the variable R^{k} is C₁ alkyl. In some embodiments of Formula I (including the embodiments discussed above that define Ring A, R¹, R², and R³), the variable R^{k} is —CH₃.

In some embodiments of Formula I (including the embodiments discussed above that define Ring A, R¹, R², and R³), the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3 —OH groups) groups.

In some embodiments of Formula I (including the embodiments discussed above that define Ring A, R¹, R², and R³), the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₆ alkyl, C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups. In some embodiments of Formula I (including the embodiments discussed above that define Ring A, R¹, R², and R³), the C₃-C₆ carbocyclyl, benzyl, and C₆ aryl of any one of R^{k} are each optionally substituted with C₁-C₆ alkyl, wherein the C₁-C₆ alkyl is optionally substituted by 1 to 3 —OH groups.

In some embodiments of Formula I (including the embodiments discussed above that define Ring A, R¹, R², and R³), the 5- to 10-membered heteroaryl and 5- to 10-membered heterocyclyl of any one of R^{k} are each optionally substituted with 1 to 3 groups independently chosen from halogen, cyano, —C(═O)CH₃, —NH₂, —OH, C₁-C₄ alkyl, C₁-C₄ haloalkyl, C₃-C₆ cycloalkyl, and C₁-C₄ alkoxy group. In some embodiments of Formula I (including the embodiments discussed above that define Ring A, R¹, R², and R³), the 5- to 10-membered heteroaryl and 5- to 10-membered heterocyclyl of any one of R^{k} are optionally substituted with C₁-C₄ alkyl, wherein the C₁-C₄ alkyl is optionally substituted by 1 to 3 —OH groups.

In some embodiments of Formula I (including the embodiments discussed above that define Ring A, R¹, R², and R³), the variable R^{k}, for each occurrence, is independently chosen from halogen, cyano, oxo, —(CH₂),C(═O)NH₂, —NH₂, —NH(C₁-C₄ alkyl), —N(C₁-C₄ alkyl)₂, C₁-C₆ alkyl, C₁-C₆ alkoxy, —C(═O)R^{k}, —S(═O)ₚR^{k}, —OR^{k}, C₃-C₆ cycloalkyl, and 5- to 10-membered heterocyclyl groups. In some embodiments of Formula I (including the embodiments discussed above that define Ring A, R¹, R², and R³), the C₁-C₆ alkyl, the C₁-C₆ alkoxy, and the 5- to 10-membered heterocyclyl of any one of R^{m} are optionally substituted with 1 to 3 groups independently chosen from halogen, cyano, —OH, and C₁-C₄ alkoxy groups.

In some embodiments of Formula I (including the embodiments discussed above that define Ring A, R¹, R², and R³), the variable R⁴ is

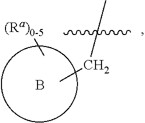

wherein Ring B is chosen from wherein R^{a} is chosen from halogen, C₁-C₄ alkyl, and C₁-C₄ alkoxy.

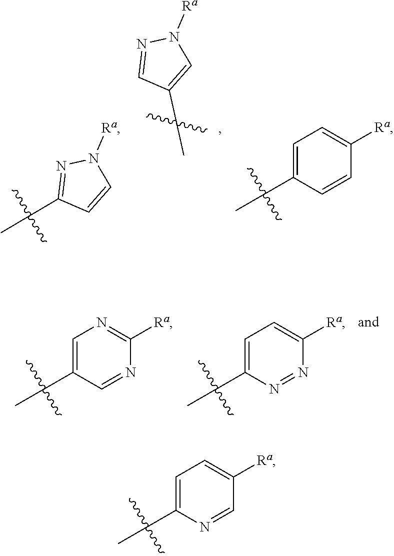

In some embodiments of Formula I (including the embodiments discussed above that define Ring A, R¹, R², and R³), the variable R⁴ is

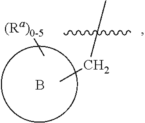

wherein Ring B is chosen from

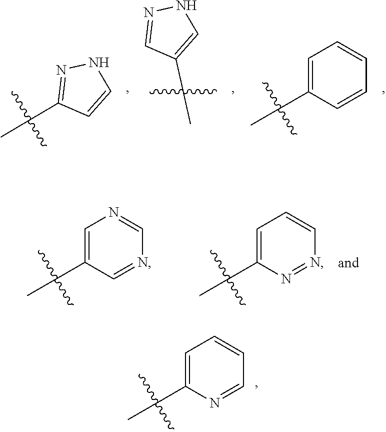 [SMILES: *c1cc[nH]n1 ; *c1cn[nH]c1 ; *c1ccccc1 ; *c1cncnc1 ; *c1cccnn1 ; *c1ccccn1]

and R^{a} is absent.

In some embodiments of Formula I (including the embodiments discussed above that define Ring A, R¹, R², and R³), the variable R⁴ is chosen from:

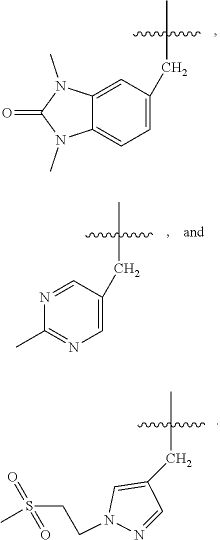

In some embodiments of Formula I (including the embodiments discussed above that define Ring A, R¹, R², R³, and R⁴), the variable R⁵ is OH. In some embodiments of Formula I (including the embodiments discussed above that define Ring A, R¹, R², R³, and R⁴), the variable R⁵ is chosen from halogen, cyano, C₁-C₆ alkyl, C₁-C₆ haloalkyl, —(CH₂),C(═O)NR⁵R^{p}, —NRⁿR^{o}, —NR^{h}C(═O)R^{p}, —NR^{h}S(═O)ₚR^{P}, —(CH₂)ₙOR^{p}, —S(═O)ₚR^{P}, —S(═O)ₚNRⁿR^{o}, —OS(═O)ₚNRⁿR^{o}, and —(CH₂)ₙC(═O)OR^{P} groups, wherein the variables Rⁿ and R^{o}, for each occurrence, are each independently chosen from hydrogen and C₁-C₄ alkyl groups; and wherein the variable R^{p}, for each occurrence, is independently chosen from hydrogen, C₁-C₄ alkyl, and C₁-C₄haloalkyl groups. In some embodiments of Formula I (including the embodiments discussed above that define Ring A, R¹, R², R³, and R⁴), the variable R⁵ is —(CH₂)ₙOR^{p}. In some embodiments of Formula I (including the embodiments discussed above that define Ring A, R¹, R², R³, and R⁴), the variable n is 0. In some embodiments of Formula I (including the embodiments discussed above that define Ring A, R¹, R², R³, and R⁴), the variable R^{p} is hydrogen.

In some embodiments of Formula I (including the embodiments discussed above that define Ring A, R¹, R², R³, R⁴, and R⁵), the variable m is an integer chosen from 0, 1, 2, 3, 4, and 5. In some embodiments of Formula I (including the embodiments discussed above that define Ring A, R¹, R², R³, R⁴, and R⁵), the variable m is an integer chosen from 0, 1, and 2. In some embodiments of Formula I (including the embodiments discussed above that define Ring A, R¹, R², R³, R⁴, and R⁵), the variable m is 0. In some embodiments of Formula I (including the embodiments discussed above that define Ring A, R¹, R², R³, R⁴, and R⁵), the variable m is 1. In some embodiments of Formula I (including the embodiments discussed above that define Ring A, R¹, R², R³, R⁴, and R⁵), the variable m is 2.

In some embodiments of Formula I (including the embodiments discussed above that define Ring A, R¹, R², R³, R⁴, R⁵, and m), the variable n is an integer chosen from 0, 1, and 2. In some embodiments of Formula I (including the embodiments discussed above that define Ring A, R¹, R², R³, R⁴, R⁵, and m), n is 0.

In some embodiments of Formula I (including the embodiments discussed above that define Ring A, R¹, R², R³, R⁴, R⁵, m, and n), the variable p, for each occurrence, is an integer independently chosen from 1 and 2. In some embodiments of Formula I (including the embodiments discussed above that define the variables Ring A, R¹, R², R³, R⁴, R⁵, m, and n), p is 2.

In some embodiments of Formula I (including the embodiments discussed above that define the variables Ring A, R¹, R², R³, R⁴, R⁵, m, n, and p), the variables q and r, for each occurrence, are each an integer independently chosen from 0, 1, 2, and 3.

In some embodiments of Formula I, at least one of R² and R³ is hydrogen and the other is chosen from C₁-C₄ alkyl groups. In these embodiments, the compound of Formula I is a compound represented by the enantiomeric structures of Formulae IIa and IIb:

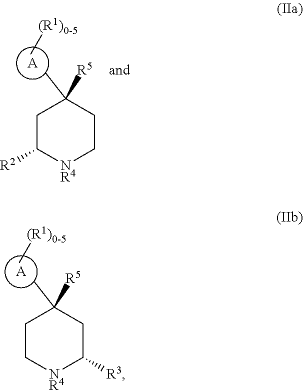

or a tautomer thereof, a deuterated derivative of those compounds and tautomers, or a pharmaceutically acceptable salt of any of the foregoing, wherein R² and R³ are chosen from C₁-C₄ alkyl groups, and Ring A, R¹, R⁴, and R⁵ are as defined above for Formula I.

In some embodiments, the at least one compound, tautomer, deuterated derivative, or pharmaceutically acceptable salt of the disclosure is chosen from Compounds 1 to 26 depicted in Table 1, a tautomer thereof, a deuterated derivative of that compound or tautomer, or a pharmaceutically acceptable salt of any of the foregoing. A wavy line in a compound in Table 1 (i.e., [symbol] ) depicts a bond between two atoms and indicates a position of mixed stereochemistry for a collection of molecules, such as a racemic mixture, cis/trans isomers, or (E)/(Z) isomers. An asterisk adjacent to an atom (e.g.,

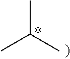

in a compound in Table 1, indicates a chiral position in the molecule.

In some embodiments, the compound of Formula I is selected from the compounds presented in Table 1 below, tautomers of those compounds, deuterated derivatives of those compounds and tautomers, and pharmaceutically acceptable salts of any of the foregoing.

TABLE 1
Compounds 1 to 26

| | |
|---|---|
| 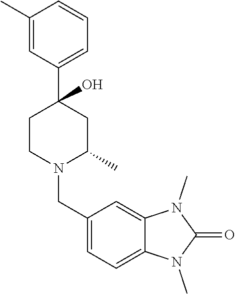 [SMILES: Cc1cccc([C@@]2(O)CCN(Cc3ccc4c(c3)n(C)c(=O)n4C)[C@@H](C)C2)c1] | 1 |
| 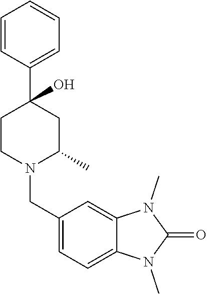 [SMILES: C[C@H]1C[C@@](O)(c2ccccc2)CCN1Cc1ccc2c(c1)n(C)c(=O)n2C] | 2 |
| 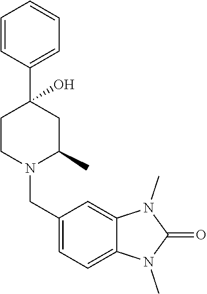 [SMILES: C[C@@H]1C[C@](O)(c2ccccc2)CCN1Cc1ccc2c(c1)n(C)c(=O)n2C] | 3 |
| 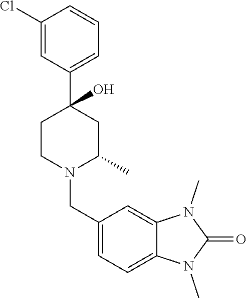 [SMILES: C[C@H]1C[C@@](O)(c2cccc(Cl)c2)CCN1Cc1ccc2c(c1)n(C)c(=O)n2C] | 4 |
| 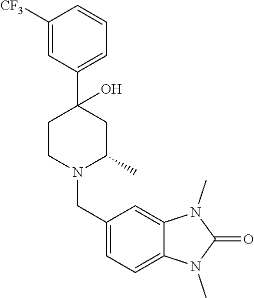 [SMILES: C[C@H]1CC(O)(c2cccc(C(F)(F)F)c2)CCN1Cc1ccc2c(c1)n(C)c(=O)n2C] | 5 |
| 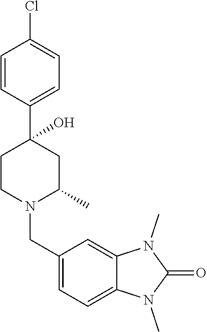 [SMILES: C[C@H]1C[C@](O)(c2ccc(Cl)cc2)CCN1Cc1ccc2c(c1)n(C)c(=O)n2C] | 6 |
| 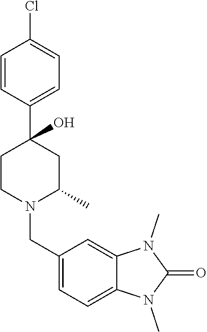 [SMILES: C[C@H]1C[C@@](O)(c2ccc(Cl)cc2)CCN1Cc1ccc2c(c1)n(C)c(=O)n2C] | 7 |
| 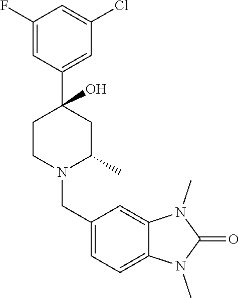 [SMILES: C[C@H]1C[C@@](O)(c2cc(F)cc(Cl)c2)CCN1Cc1ccc2c(c1)n(C)c(=O)n2C] | 8 |
| 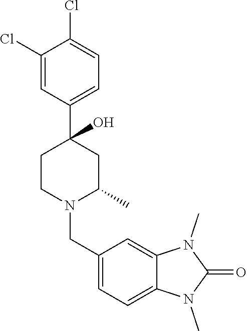 [SMILES: C[C@H]1C[C@@](O)(c2ccc(Cl)c(Cl)c2)CCN1Cc1ccc2c(c1)n(C)c(=O)n2C] | 9 |
| 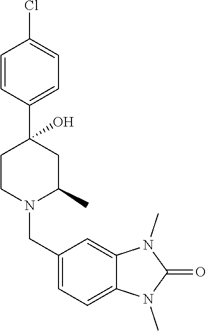 [SMILES: C[C@@H]1C[C@](O)(c2ccc(Cl)cc2)CCN1Cc1ccc2c(c1)n(C)c(=O)n2C] | 10 |
| 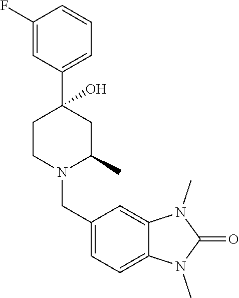 [SMILES: C[C@@H]1C[C@](O)(c2cccc(F)c2)CCN1Cc1ccc2c(c1)n(C)c(=O)n2C] | 11 |
| 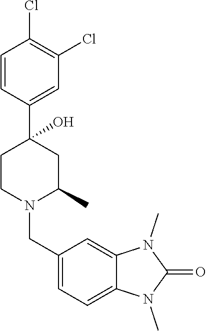 [SMILES: C[C@@H]1C[C@](O)(c2ccc(Cl)c(Cl)c2)CCN1Cc1ccc2c(c1)n(C)c(=O)n2C] | 12 |
| 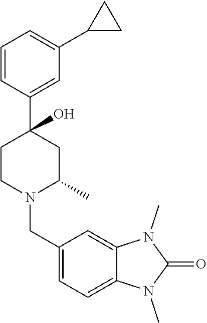 [SMILES: C[C@H]1C[C@@](O)(c2cccc(C3CC3)c2)CCN1Cc1ccc2c(c1)n(C)c(=O)n2C] | 13 |
| 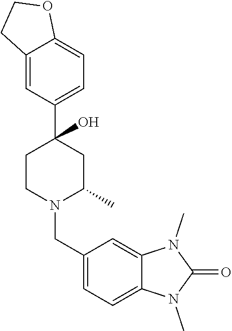 [SMILES: C[C@H]1C[C@@](O)(c2ccc3c(c2)CCO3)CCN1Cc1ccc2c(c1)n(C)c(=O)n2C] | 14 |
| 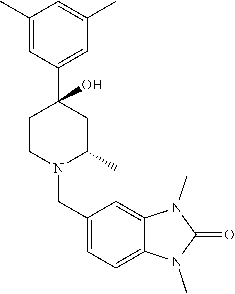 [SMILES: Cc1cc(C)cc([C@@]2(O)CCN(Cc3ccc4c(c3)n(C)c(=O)n4C)[C@@H](C)C2)c1] | 15 |
| 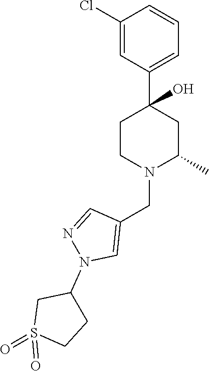 [SMILES: C[C@H]1C[C@@](O)(c2cccc(Cl)c2)CCN1Cc1cnn(C2CCS(=O)(=O)C2)c1] | 16 |
| 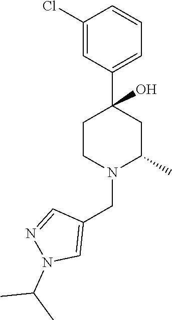 [SMILES: CC(C)n1cc(CN2CC[C@](O)(c3cccc(Cl)c3)C[C@@H]2C)cn1] | 17 |
| 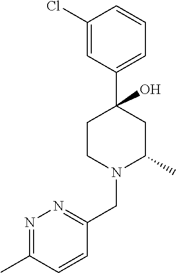 | 18 |
| 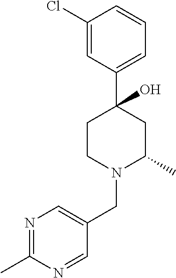 | 19 |
| 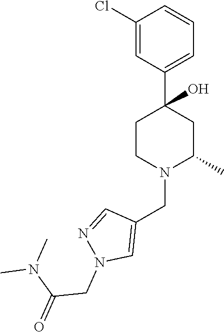 | 20 |
| 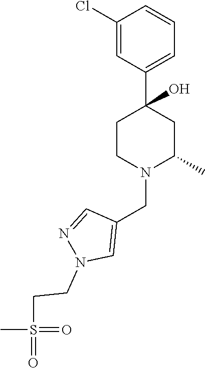 | 21 |
| 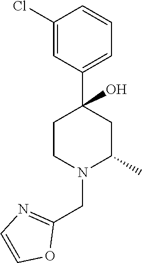 [SMILES: C[C@H]1C[C@@](O)(c2cccc(Cl)c2)CCN1Cc1ncco1] | 22 |
| 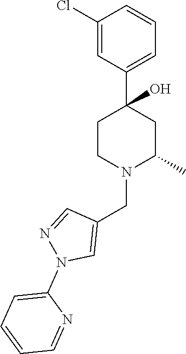 [SMILES: C[C@H]1C[C@@](O)(c2cccc(Cl)c2)CCN1Cc1cnn(-c2ccccn2)c1] | 23 |
| 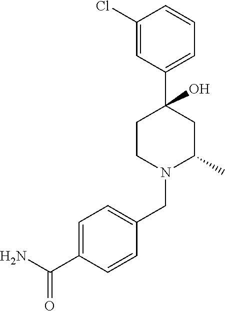 [SMILES: C[C@H]1C[C@@](O)(c2cccc(Cl)c2)CCN1Cc1ccc(C(N)=O)cc1] | 24 |
| 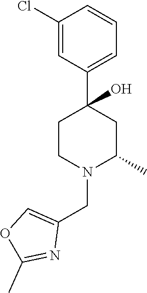 [SMILES: Cc1nc(CN2CC[C@](O)(c3cccc(Cl)c3)C[C@@H]2C)co1] | 25 |
| 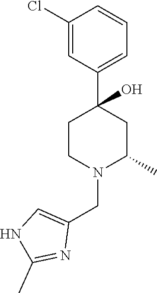 [SMILES: Cc1nc(CN2CC[C@](O)(c3cccc(Cl)c3)C[C@@H]2C)c[nH]1] | 26 |

Some embodiments of the disclosure include derivatives of Compounds 1 to 26 or compounds of Formulae I, IIa, and IIb, tautomers thereof, deuterated derivatives of those compounds or tautomers, or pharmaceutically acceptable salts of any of the foregoing. In some embodiments, the derivatives are silicon derivatives in which at least one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by silicon. In some embodiments, the derivatives are boron derivatives, in which at least one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by boron. In other embodiments, the derivatives are phosphorus derivatives, in which at least one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by phosphorus.

In some embodiments, the derivative is a silicon derivative in which one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by silicon or a silicon derivative (e.g., —Si(CH₃)₂— or —Si(OH)₂—). The carbon replaced by silicon may be a non-aromatic carbon. In other embodiments, a fluorine has been replaced by silicon derivative (e.g., —Si(CH₃)₃). In some embodiments, the silicon derivatives of the disclosure may include one or more hydrogen atoms replaced by deuterium. In some embodiments, a silicon derivative of compound, tautomer, deuterated derivative, or pharmaceutically acceptable salt chosen from Compounds 1 to 26 or compounds of Formulae I, IIa, and IIb, a tautomer thereof, a deuterated derivative of that compound or tautomer, or a pharmaceutically acceptable salt of any of the foregoing, may have silicon incorporated into a heterocycle ring.

In some embodiments, the derivative is a boron derivative in which one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by boron or a boron derivative.

In some embodiments, the derivative is a phosphorus derivative in which one carbon atom in a compound, tautomer, deuterated derivative, or pharmaceutically acceptable salt chosen from Compounds 1 to 26 or compounds of Formulae I, IIa, and IIb, tautomers thereof, deuterated derivatives of those compounds or tautomers, and pharmaceutically acceptable salts of any of the foregoing, has been replaced by phosphorus or a phosphorus derivative.

Another aspect of the disclosure provides pharmaceutical compositions comprising at least one compound, tautomer, deuterated derivative, or pharmaceutically acceptable salt according to any one formula chosen from Formulae I, IIa, and IIb, and Compounds 1 to 26, tautomers thereof, deuterated derivatives of those compounds or tautomers, and pharmaceutically acceptable salts of any of the foregoing. In some embodiments, the pharmaceutical composition comprising at least one compound, tautomer, deuterated derivative, or pharmaceutically acceptable salt chosen from Formulae I, IIa, and IIb, Compounds 1 to 26, tautomers thereof, deuterated derivatives of those compounds or tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent. In some embodiments, a pharmaceutical composition comprising at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21′ edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

### Use of Compounds and Compositions

In some embodiments of the disclosure, the compounds and the pharmaceutical compositions described herein are used to treat FSGS and/or NDKD. In some embodiments, FSGS is mediated by APOL1. In some embodiments, NDKD is mediated by APOL1.

In some embodiments of the disclosure, the compounds and the pharmaceutical compositions described herein are used to treat cancer. In some embodiments, the cancer is mediated by APOL1.

In some embodiments of the disclosure, the compounds and the pharmaceutical compositions described herein are used to treat pancreatic cancer. In some embodiments, the pancreatic cancer is mediated by APOL1.

In some embodiments, the methods of the disclosure comprise administering to a patient in need thereof at least one compound, tautomer, deuterated derivative, or pharmaceutically acceptable salt chosen from compounds of Formulae I, IIa, and IIb, tautomers thereof, deuterated derivatives of those compounds and tautomers, and pharmaceutically acceptable salts of any of the foregoing. In some embodiments, the compound, tautomer, deuterated derivative, or pharmaceutically acceptable salt is chosen from Compounds 1 to 26, tautomer thereof, deuterated derivatives of those compounds or tautomers, and pharmaceutically acceptable salts of any of the foregoing. In some embodiments, said patient in need thereof possesses APOL1 genetic variants, i.e., G1: S342G:I384M and G2: N388del:Y389del.

Another aspect of the disclosure provides methods of inhibiting APOL1 activity comprising contacting said APOL1 with at least one compound, tautomer, deuterated derivative, or pharmaceutically acceptable salt chosen from compounds of Formulae I, IIa, and IIb, tautomers thereof, deuterated derivatives of those compounds or tautomers, and pharmaceutically acceptable salts of any of the foregoing. In some embodiments, the methods of inhibiting APOL1 activity comprise contacting said APOL1 with at least one compound, tautomer, deuterated derivative, or pharmaceutically acceptable salt chosen from Compounds 1 to 26, tautomers thereof, deuterated derivatives of those compounds or tautomers, and pharmaceutically acceptable salts of any of the foregoing.

### Examples

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae I, IIa, and IIb, Compounds 1 to 26, tautomers thereof, deuterated derivatives of those compounds and tautomers, and pharmaceutically acceptable salts of any of the foregoing, the following abbreviations are used:

### Abbreviations

n-BuLi=n-butyllithium
DCM=dichloromethane
DMF=dimethylformamide
DMSO=dimethyl sulfoxide
EtOAc=ethyl acetate
Et₃N=triethylamine
HPLC=high-performance liquid chromatography
KI=potassium iodide
K₂CO₃=potassium carbonate
LaCl₃·(LiCl)₂=lanthanum (III) chloride bis (lithium chloride) complex
LCMS=liquid chromatography mass spectrometry
MeCN=acetonitrile
MeOH=methanol
NaHCO₃=sodium bicarbonate
Na₂SO₄=sodium sulfate
NH₄Cl=ammonium chloride
NMR=nuclear magnetic resonance
PMB-Cl=p-methoxybenzyl chloride; 4-methoxybenzyl chloride
rt=room temperature
SFC=supercritical fluid chromatography
SiO₂=silicon dioxide
tet=tetracycline
THF=tetrahydrofuran Example 1. Synthesis of Compounds

All the specific and generic compounds, and the intermediates disclosed for making those compounds, are considered to be part of the disclosure disclosed herein.

### Compound 1

### 5-[[(2S,4R)-4-hydroxy-2-methyl-4-(m-tolyl)-1-piperidyl]methyl]-1,3-dimethyl-benzimidazol-2-one (1)

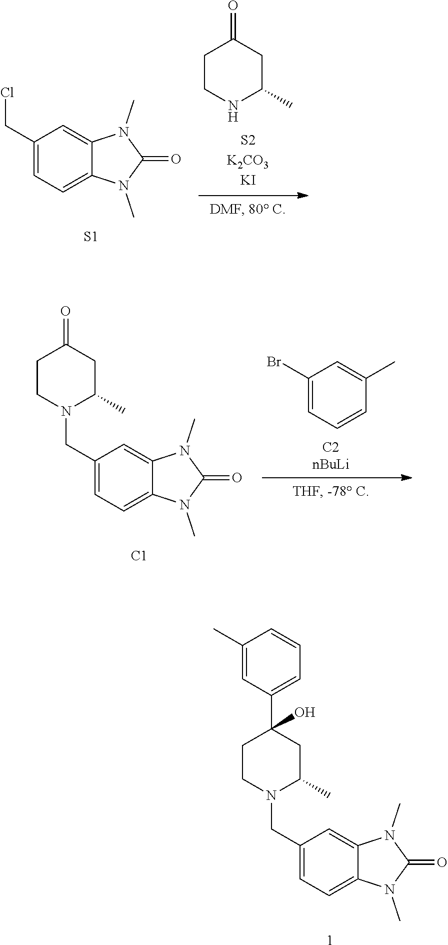

### Step 1. Synthesis of 1,3-dimethyl-5-[[(2S)-2-methyl-4-oxo-1-piperidyl]methyl]benzimidazol-2-one (C)

To a solution of 5-(chloromethyl)-1,3-dimethyl-benzimidazol-2-one (Si) (5.5 g, 22.2 mmol) in DMF (15 mL), (2S)-2-methylpiperidin-4-one (S2) (7.5 g, 59.9 mmol) and K₂CO₃ (15.3 g, 110 mmol) and KI (365 mg, 2.2 mmol) were added, at room temperature, the reaction mixture was allowed to warm to 80° C. and stirred at 80° C. for 16 h. At this time, the reaction mixture was poured into ice-cold water (500 mL) and extracted with ethyl Acetate (3×200 mL), washed with brine (60 mL), dried over anhydrous sodium sulfate, filtered and concentrated under reduced pressure. The crude was purified by column chromatography (100-200 silica gel, eluted with 5% DCM in Methanol). The product-containing fractions were pooled and concentrated to afford the title compound C₁ (1.2 g, 16%) as a yellow gum. ¹H NMR (300 MHz, DMSO-d₆) δ 7.12 (s, 1H), 7.09 (m, 2H), 3.93-3.90 (m, 1H), 3.93 (s, 1H), 3.55-3.51 (m, 1H), 3.35-3.30 (m, 6H), 3.01-2.99 (m, 1H), 2.88-2.74 (m, 1H), 2.56-2.51 (m, 1H), 2.49 (m, 1H), 2.30-2.27 (m, 1H), 2.18-2.14 (m, 1H), 1.08 (s, 3H).

### Step 2. Synthesis of 5-[[(2S,4R)-4-hydroxy-2-methyl-4-(m-tolyl)-1-piperidyl]methyl]-1,3-dimethyl-benzimidazol-2-one (1)

To a solution of 1-bromo-3-methyl-benzene (C₂) (300 mg, 1.7 mmol) and C₁ (500 mg, 1.6 mmol) in THF (10 mL), n-BuLi (1.4 mL of 2.5 M, 3.5 mmol) was added at −78° C. The reaction temperature was warmed to rt over 2 h. At this time, the reaction mixture was quenched with sat NH₄Cl solution, extracted with EtOAc (2×80 mL), and the combined organics were washed with brine (40 mL), dried over Na₂SO₄ and evaporated solvent to get a crude. The crude compound was purified by Prep-HPLC (Mobile phase A: 10 mM Ammonium bicarbonate (Aq) Mobile phase B: Acetonitrile, Column: XbridgeC18, 250 mm×19 mm×5 m, Flow: 14 ml/min, 15-98% MeCN in water, rt). The combined fractions were concentrated under reduced pressure and purified by preparatory SFC (Chiralpak © IC, 250 mm×30 mm×5 m, Mobile phase: 45% 30 mM ammonia in methanol, 65% carbon dioxide, 70 mL/min, 100.0 bar, 30° C.). The product-containing fractions were pooled and concentrated to yield the title compound 1 (25 mg, 4%) as an off-white solid. ¹H NMR (400 MHz, DMSO-d₆) δ 7.28-7.24 (m, 2H), 7.19 (t, J=7.2 Hz, 1H), 7.059-6.99 (m, 4H), 4.76 (s, 1H), 3.85 (d, J=13.2 Hz, 1H), 3.32 (s, 6H), 3.20 (d, J=13.2 Hz, 1H), 2.73-2.69 (m, 1H), 2.60-2.51 (m, 1H), 2.30 (s, 3H), 2.24-2.20 (m, 1H), 2.11-2.07 (m, 1H), 2.03-1.98 (m, 1H), 1.63-1.58 (m, 2H), 1.20 (d, J=6.4 Hz, 23H). LCMS m/z 380.2 [M+H]⁺.

Compounds 2-16 were prepared in analogous fashion to compound 1, using the appropriately chosen 2-(R)methylpiperidone or 2-(S)methylpiperidone starting material as reagent S2.

TABLE 2
Structure and Physiochemical Data for Compounds 2-15

| Compound | Aryl Halide | Structure | ¹H NMR, LCMS M + 1 |
|---|---|---|---|
| 2 | 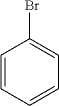 C3 | 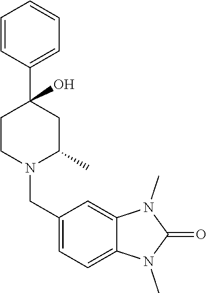 [SMILES: C[C@H]1C[C@@](O)(c2ccccc2)CCN1Cc1ccc2c(c1)n(C)c(=O)n2C] | ¹H NMR (400 MHz, DMSO-d₆) δ 7.46 (dd, J = 8.4 Hz, 1.2 Hz, 2H), 7.29 (t, J = 8 Hz, 2H), 7.20-7.16 (m, 1H), 7.08-7.02 (m, 3H), 4.75 (s, 1H), 4.13 (d, J = 12.2 Hz, 1H), 3.33 (s, 3H), 3.32 (s, 3H), 3.13 (d, J = 13.2 Hz, 1H), 2.72- 2.67 (m, 1H), 2.56-2.49 (m, 1H), 2.43- 2.37 (m, 1H), 1.84-1.79 (m, 1H), 1.65- 1.63 (m, 2H), 1.50-1.46 (m, 1H), 1.15 (d, J = 6 Hz, 3H). LCMS m/z 366.23 [M + H]⁺ |
| 3 | 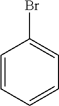 C3 | 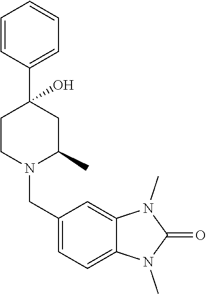 [SMILES: C[C@@H]1C[C@](O)(c2ccccc2)CCN1Cc1ccc2c(c1)n(C)c(=O)n2C] | ¹H NMR (400 MHz, DMSO-d₆) δ 7.46 (d, J = 7.2 Hz, 2H), 7.29 (t, J = 7.6 Hz, 2H), 7.20-7.16 (m, 1H), 7.08-7.05 (m, 3H), 4.75 (s, 1H), 4.13 (d, J = 13.2 Hz, 1H), 3.33 (s, 6H), 3.13 (d, J = 13.2 Hz, 1H), 2.72-2.66 (m, 1H), 2.56-2.49 (m,1H), 2.43-2.32 (m, 1H), 1.84-1.75 (m, 1H), 1.66-1.63 (m, 2H), 1.49 (d, J = 13.2 Hz, 1H), 1.15 (d, J = 6.4 Hz, 3H). LCMS m/z 366.2 [M + H]⁺ |
| 4 | 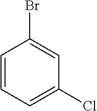 C4 | 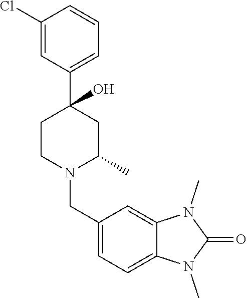 [SMILES: C[C@H]1C[C@@](O)(c2cccc(Cl)c2)CCN1Cc1ccc2c(c1)n(C)c(=O)n2C] | ¹H NMR (400 MHz, DMSO-d₆) δ 7.50 (t, J = 2 Hz, 1H ), 7.41-7.39 (m, 1H), 7.33 (t, J = 8 Hz, 1H), 7.27-7.24 (m, 1H), 7.08-7.02 (m, 3H), 4.94 (s, 1H), 4.12 (d, J = 13.2 Hz, 1H), 3.33 (s, 3H), 3.31 (s, 3H), 3.13 (d, J = 12.8 Hz, 1H ), 2.71-2.66 (m, 1H), 2.56- 2.49 (m, 1H), 2.41 (t, J = 2.4 Hz, 1H), 1.79 (dd, J = 4.4 Hz, J = 12.8 Hz, 1H), 1.64 (q, J = 13.6 Hz, 2H), 1.47 (dd, J = 2 Hz, J = 13.2 Hz, 1H), 1.15 (d, J = 6.4 Hz, 3H). LCMS m/z 400.19 [M + H]⁺ |
| 5 | 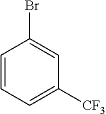 C5 | 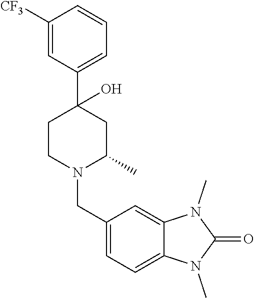 [SMILES: C[C@H]1CC(O)(c2cccc(C(F)(F)F)c2)CCN1Cc1ccc2c(c1)n(C)c(=O)n2C] | ¹H NMR (300 MHz, Chloroform-d) δ 7.84-7.42 (m, 4H), 7.16-6.84 (m, 3H), 4.15 (dd, J = 89.1, 13.1 Hz, 1H), 3.45 (dd, J = 6.4, 3.5 Hz, 6H), 3.25 (dd, J = 31.8, 13.1 Hz, 1H), 2.96- 2.70 (m, 2H), 2.50-2.36 (m, 1H), 2.30-2.02 (m, 1H), 2.01-1.75 (m, 2H), 1.67 (d, J = 13.7 Hz, 1H), 1.31 (dd, J = 11.0, 6.3 Hz, 3H).; 19F NMR (282 MHz, CDCl₃) δ −62.49, −62.50.; LCMS m/z 434.1 [M + H]⁺ 1.4:1 mixture of cis and trans isomers |
| 6^{a} | 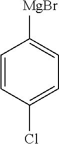 C6 | 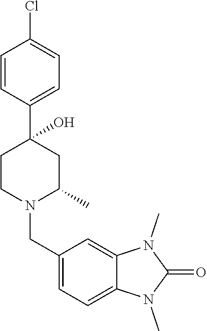 [SMILES: C[C@H]1C[C@](O)(c2ccc(Cl)cc2)CCN1Cc1ccc2c(c1)n(C)c(=O)n2C] | ¹H NMR (400 MHz, DMSO-d₆) δ 7.50 (d, J = 8.3 Hz, 2H), 7.39 (d, J = 8.2 Hz, 2H), 7.12 (d, J = 22.2 Hz, 3H), 5.32-4.90 (m, 2H), 4.29-3.79 (m, 2H), 3.32 (d, J = 1.9 Hz, 6H), 2.89 (s, 2H), 2.37-2.00 (m, 2H), 1.69 (s, 2H), 1.31 (s, 3H). LCMS m/z 400.38 [M + H]⁺ |
| 7^{a} | 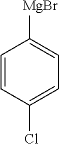 C6 | 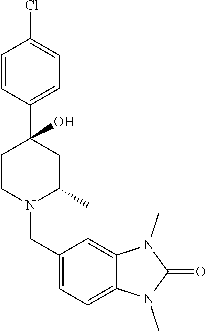 [SMILES: C[C@H]1C[C@@](O)(c2ccc(Cl)cc2)CCN1Cc1ccc2c(c1)n(C)c(=O)n2C] | ¹H NMR (400 MHz, DMSO-d₆) δ 7.48 (d, J = 8.4 Hz, 2H), 7.36 (d, J = 8.4 Hz, 2H), 7.20-7.01 (m, 3H), 5.04 (s, 1H), 4.24 (s, 1H), 3.33 (d, J = 4.9 Hz, 7H), 2.67 (d, J = 29.9 Hz, 1H), 1.93-1.46 (m, 4H), 1.22 (s, 3H). LCMS m/z 400.29 [M + H]⁺ |
| 8^{a} | 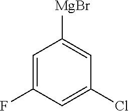 C7 | 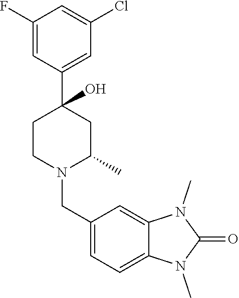 [SMILES: C[C@H]1C[C@@](O)(c2cc(F)cc(Cl)c2)CCN1Cc1ccc2c(c1)n(C)c(=O)n2C] | ¹H NMR (400 MHz, DMSO-d₆) δ 7.35 (d, J = 1.7 Hz, 1H), 7.25 (dd, J = 9.4, 1.7 Hz, 2H), 7.12-7.01 (m, 3H), 5.18 (s, 1H), 4.17 (d, J = 13.2 Hz, 1H), 3.32 (d, J = 5.2 Hz, 6H), 3.28- 3.20 (m, 1H), 2.81-2.72 (m, 1H), 2.63-2.54 (m, 1H), 1.84 (t, J = 12.6 Hz, 1H), 1.77-1.44 (m, 3H), 1.18 (d, J = 6.1 Hz, 3H). LCMS m/z 418.31 [M + H]⁺ |
| 9^{a} | 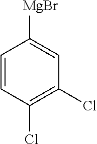 C8 | 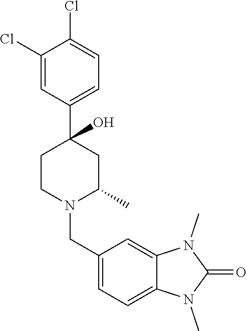 [SMILES: C[C@H]1C[C@@](O)(c2ccc(Cl)c(Cl)c2)CCN1Cc1ccc2c(c1)n(C)c(=O)n2C] | ¹H NMR (400 MHz, DMSO-d₆) δ 7.67 (d, J = 2.1 Hz, 1H), 7.56 (d, J = 8.4 Hz, 1H), 7.44 (dd, J = 8.4, 2.2 Hz, 1H), 7.06 (dd, J = 14.9, 8.0 Hz, 3H), 5.11 (d, J = 3.2 Hz, 1H), 4.14 (d, J = 13.1 Hz, 1H), 3.33 (d, J = 5.2 Hz, 6H), 3.15 (d, J = 13.0 Hz, 1H), 2.69 (s, 1H), 2.38 (t, J = 12.1 Hz, 1H), 1.89- 1.75 (m, 1H), 1.73-1.41 (m, 3H), 1.17 (d, J = 6.1 Hz, 3H). LCMS m/z 434.29 [M + H]⁺ |
| 10 | 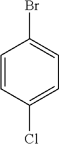 C9 | 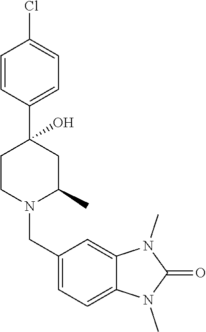 [SMILES: C[C@@H]1C[C@](O)(c2ccc(Cl)cc2)CCN1Cc1ccc2c(c1)n(C)c(=O)n2C] | ¹H NMR (400 MHz, DMSO-d₆) δ 7.48 (d, J = 8.4 Hz, 2H), 7.34 (d, J = 8.4 Hz, 2H), 7.08-7.02 (m, 3H), 4.89 (brs, 1H), 4.12 (d, J = 13.2 Hz, 1H), 3.31 (s, 6H), 3.11 (d, J = 12.8 Hz, 1H), 2.71 (brs, 1H), 2.55 (brs, 1H), 2.41 (m, 1H), 2.35 (m, 1H), 1.82 (m, 1H), 1.67 (brs, 1H), 1.64 (m, 1H), 1.48 (s, 3H). LCMS m/z 400.1 [M + H]⁺ |
| 11 | 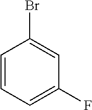 C10 | 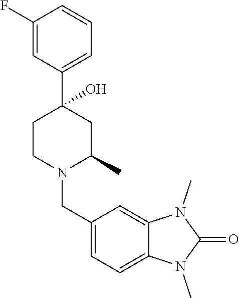 [SMILES: C[C@@H]1C[C@](O)(c2cccc(F)c2)CCN1Cc1ccc2c(c1)n(C)c(=O)n2C] | ¹H NMR (400 MHz, DMSO-d₆) δ 7.49 (d, J = 8.4 Hz, 2H), 7.35 (d, J = 8.4 Hz, 2H), 7.05-6.99 (m, 3H), 4.94 (brs, 1H), 3.85 (d, J = 13.2 Hz, 1H), 3.31-3.25 (s, 6H), 3.19 (d, J = 12.8 Hz, 1H), 2.71 (brs, 1H), 2.55 (brs, 1H), 2.23-2.20 (m, 3H), 2.09-1.97 (m, 1H), 1.63 (m, 1H), 1.23 (brs, 3H). LCMS m/z 384.2 [M + H]⁺ |
| 12 | 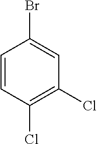 C11 | 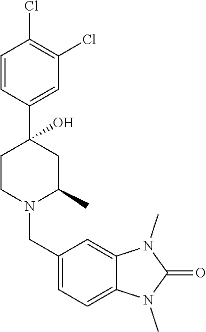 [SMILES: C[C@@H]1C[C@](O)(c2ccc(Cl)c(Cl)c2)CCN1Cc1ccc2c(c1)n(C)c(=O)n2C] | ¹H NMR (400 MHz, DMSO-d₆) δ 7.67 (d, J = 2.0 Hz, 1H), 7.55 (d, J = 8.4 Hz, 1H), 7.45-7.42 (m, 1H), 7.07- 7.02 (m, 3H), 4.89 (brs, 1H), 4.12 (d, J = 12.8 Hz, 1H), 3.32 (s, 6H), 3.14 (m, 1H), 2.70-2.68 (m, 1H), 2.56 (m, 1H), 2.41 (m, 1H), 2.37 (m, 1H), 1.84 (m, 1H), 1.67 (brs, 1H), 1.64 (m, 1H), 1.48 (s, 3H). LCMS m/z 434.1 [M + H]⁺ |
| 13 | 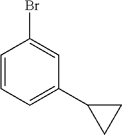 C12 | 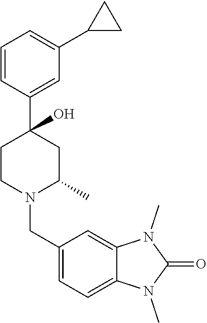 [SMILES: C[C@H]1C[C@@](O)(c2cccc(C3CC3)c2)CCN1Cc1ccc2c(c1)n(C)c(=O)n2C] | ¹H NMR (400 MHz, DMSO-d₆) δ 7.23-7.15 (m, 3H), 7.06-6.85 (m, 3H), 6.86 (d, J = 7.2 Hz, 1H), 4.76 (s, 1H), 3.85-3.82 (d, J = 13.2 Hz, 1H), 3.36- 3.20 (m, 7H), 2.71 (m, 1H), 2.50 (s, 1H), 2.23-1.89 (m, 4H), 1.63-1.58 (m, 2H), 1.20 (d, J = 6 Hz, 3H), 0.94-0.90 (m, 2H), 0.65-0.61 (m, 2H). LCMS m/z 406.2 [M + H]⁺ |
| 14 | 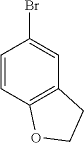 C13 | 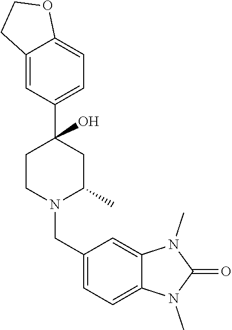 [SMILES: C[C@H]1C[C@@](O)(c2ccc3c(c2)CCO3)CCN1Cc1ccc2c(c1)n(C)c(=O)n2C] | ¹H NMR (400 MHz, DMSO-d₆) δ 7.31 (s, 1H), 7.15 (d, J = 8.0 Hz, 1H), 7.05-7.02 (m, 3H), 6.64 (d, J = 8.0 Hz, 1H), 4.61 (s, 1H), 4.48 (t, J = 8.8 Hz, 2H), 4.12 (d, J = 13.2 Hz, 1H), 3.31 (s, 6H), 3.15-3.10 (m, 3H), 2.69- 2.64 (m, 1H), 2.53 (brs, 1H), 2.40- 2.35 (m, 1H), 1.79-1.73 (m, 1H), 1.62- 1.60 (m, 2H), 1.52-1.45 (m, 1H), 1.11 (d, J = 8.0 Hz, 3H). LCMS m/z 408.2 [M + H]⁺ |
| 15 | 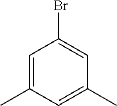 C14 | 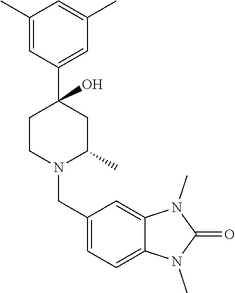 [SMILES: Cc1cc(C)cc([C@@]2(O)CCN(Cc3ccc4c(c3)n(C)c(=O)n4C)[C@@H](C)C2)c1] | ¹H NMR (400 MHz, DMSO-d₆) δ 7.07-7.02 (m, 5H), 6.81 (s, 1H), 4.65 (s, 1H), 4.12 (d, J = 12.8 Hz, 1H), 3.33 (m, 6H), 2.69-2.66 (m, 1H), 2.52 (brs, 1H), 2.40-2.34 (m, 1H), 2.25 (s, 6H), 1.79-171 (m, 1H), 1.64-1.57 (m, 1H), 1.45-1.43 (m, 3H), 1.14 (d, J = 6 Hz, 3H). LCMS m/z 394.43 [M + H]⁺ |

^{a}Alternate conditions: Grignard reagent 1.25 equiv, LaCl₃•(LiCl)₂ 1.25 equiv, no n-BuLi, 0° C.

### Compound 16

### (2S,4R)-4-(3-chlorophenyl)-1-[[1-(1,1-dioxothiolan-3-yl)pyrazol-4-yl]methyl]-2-methyl-piperidin-4-ol (16)

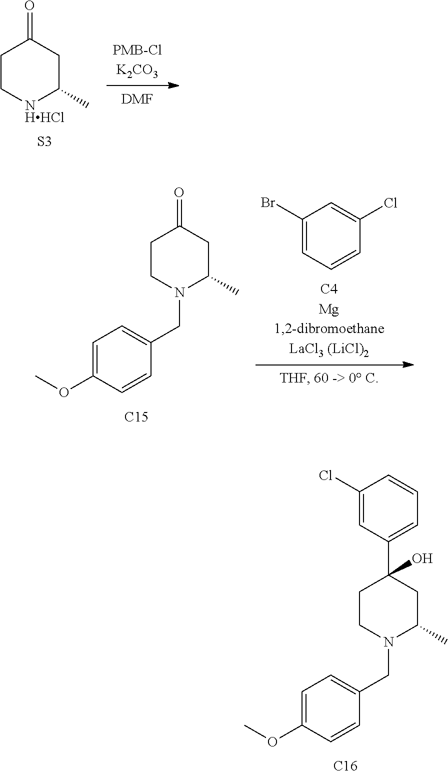

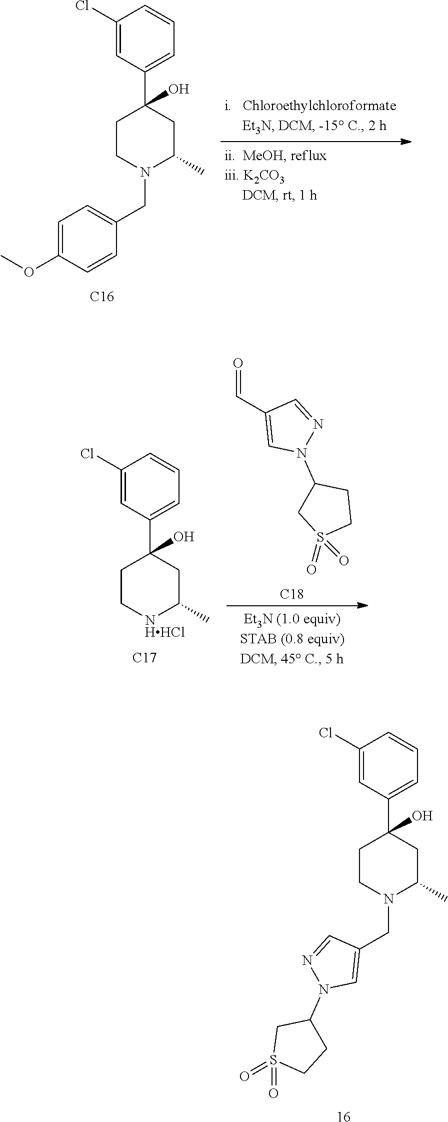

### Step 1. Synthesis of (2S)-1-[(4-methoxyphenyl)methyl]-2-methyl-piperidin-4-one (C₁₅)

To a solution of (2S)-2-methylpiperidin-4-one hydrochloride S3 (6.8 g, 45 mmol) in DMF (50 mL) was added PMB-Cl (8.6284 g, 7.5 mL, 54 mmol) followed by the addition of K₂CO₃ (34.9 g, 247.5 mmol) at room temperature. The reaction mixture was stirred at room temperature for 16 h. At this time, the reaction mixture was diluted with water (200 mL) and extracted with ethyl acetate (3×500 mL). The combined organic layers were dried over sodium sulfate and concentrated under vacuum to get crude (12 g). The crude compound was purified by column chromatography (SiO₂, 30-40% Ethyl acetate in hexane). The product-containing fractions were pooled and concentrated to afford the title compound C₁₅ (7 g, 67%) as a brown gum. ¹H NMR (300 MHz, DMSO-d₆): 7.26 (d, J=8.4 Hz, 2H), 6.89 (d, J=8.4 Hz, 2H), 3.83-3.76 (m, 1H), 3.73 (s, 3H), 3.69-3.45 (m, 1H), 2.99-2.85 (m, 2H), 2.55-2.44 (m, 2H), 2.35-2.10 (m, 3H), 1.06 (d, J=6.9 Hz, 3H). LCMS m/z 234.39 [M+H]⁺.

### Step 2. Synthesis of (2S,4R)-4-(3-chlorophenyl)-1-[(4-methoxyphenyl)methyl]-2-methyl-piperidin-4-ol (C₁₆)

To a solution of magnesium (1.6 g, 64.5 mmol) in THF (70 mL) was added 1,2-di bromo ethane (50 mg, 0.26 mmol) followed by 1-bromo-3-chloro-benzene (C₄) (13.1 g, 10 mL, 67.2 mmol) in THF (70 mL) at room temperature for 75 min. The reaction was cooled to 0° C., at which time a THF solution of Lanthanum (III) chloride bis (lithium chloride) complex (57 mL of 0.6 M, 34.200 mmol) was added over 15 min and allowed stir at 0° C. for 30 min. A solution of C₁₅ (7 g, 26.1 mmol) in THF (70 mL) was added to the reaction mixture at 0° C. and the reaction was stirred for 1 h. At this time, the reaction mixture was quenched with water (100 mL) and extracted with EtOAc (2×500 mL), and the combined organic layer was dried over sodium sulfate, filtered, and evaporated under vacuum to get a crude mixture. The crude compound was purified by column chromatography using (SiO₂, eluted with DCM to 10% MeOH in DCM). The product-containing fractions were pooled and concentrated to afford the title compound C₁₆ (3 g, 25%) as a brown gum. ¹H NMR (400 MHz, DMSO-d₆): δ 7.50-7.49 (m, 1H), 7.41-7.38 (m, 1H), 7.32 (t, J=7.6 Hz, 1H), 7.26-7.21 (m, 3H), 6.87 (d, J 8.8 Hz, 2H), 4.93 (s, 1H), 4.0 (d, J=13.2 Hz, 1H), 3.73 (s, 3H), 3.07 (d, J=13.2 Hz, 1H), 2.67-2.65 (m, 1H), 2.52-2.49 (m, 1H), 2.40-2.34 (m, 1H), 1.84-1.76 (m, 1H), 1.62-1.60 (m, 2H), 1.49-1.46 (m, 1H), 1.12 (d, J=6.0 Hz, 3H), LCMS m/z 346.47 [M+H]⁺.

### Step 3. Synthesis of (2S,4R)-4-(3-chlorophenyl)-2-methyl-piperidin-4-ol hydrochloride (C₁₇)

To a solution of C₁₆ (6.4 g, 13.9 mmol) in DCM (140 mL) were added Triethyl amine (10 mL, 68.4 mmol) and 1-Chloroethylchloroformate (7 mL, 68.2 mmol) at −15° C. The reaction mixture was cooled to −15° C. for 2 h. The reaction mixture was evaporated under vacuum to get crude residue which was redissolved in MeOH (300 mL) and refluxed for 3 h. At this time, the reaction mixture was cooled to room temperature and evaporated under reduced pressure to yield a crude brown color residue. The crude compound was dissolved in DCM (400 mL), added K₂CO₃ (30 g, 212.7 mmol) and stirred for 1 h at room temperature. The reaction was filtered and the filtrates were evaporated under vacuum to get a crude brown gum. The crude compound was purified by column chromatography (SiO₂, eluted with 0-10% MeOH in DCM) to yield a crude solid which was recrystallized from MeCN (50 mL) to afford the title compound C₁₇ (1.4 g, 38%) as a white solid. ¹H NMR (400 MHz, DMSO-d₆): δ 8.85 (brs, 2H), 7.50-7.49 (m, 1H), 7.43-7.32 (m, 3H), 5.60 (s, 1H), 3.48-3.44 (m, 1H), 3.32-3.18 (m, 2H), 2.21-2.13 (m, 1H), 2.01-1.95 (m, 1H), 1.81-1.72 (m, 2H), 1.25 (d, J=6.8 Hz, 3H), LCMS m/z 226.11 [M+H]⁺.

### Step 4. Synthesis of (2S,4R)-4-(3-chlorophenyl)-1-[[1-(1,1-dioxothiolan-3-yl)pyrazol-4-yl]methyl]-2-methyl-piperidin-4-ol (16)

To a vial containing 1-(1,1-dioxothiolan-3-yl)pyrazole-4-carbaldehyde (17.2 mg, 0.083 mmol) was added a solution of C₁₇ (15 mg, 0.057 mmol) in DCM (1 mL) and Et₃N (8 μL, 0.057 mmol). To this slurry was added sodium triacetoxyborohydride (10 mg, 0.047 mmol) and the reaction was stirred overnight. At this time, the reaction was heated to 45° C. and stirred for 5 hours. At this time, the reaction was quenched with sat. NaHCO₃ (1 mL) passed through a phase separator and extracted with DCM (1 mL) and then passed through a phase separator. The organics were concentrated under a stream of nitrogen. The concentrated crude residues were redissolved in DMSO (1 mL) for purification by reversed-phase HPLC. (Method: Waters XSelect CSH C18 OBD Prep Column; 30×150 mm, 5 micron. Gradient: Acetonitrile in Water with 10 mM Ammonium Hydroxide) to yield the title compound 16 (5.1 mg, 20%) as a white solid. H NMR (400 MHz, DMSO-d₆) δ 7.79 (s, 1H), 7.50-7.44 (m, 2H), 7.41-7.29 (m, 2H), 7.25 (d, J=7.8 Hz, 1H), 5.23 (p, J=7.7 Hz, 1H), 4.90 (s, 1H), 3.72 (dd, J=13.9, 9.8 Hz, 2H), 3.45 (d, J=6.0 Hz, 3H), 3.25 (dt, J=13.3, 8.1 Hz, 1H), 2.69-2.39 (m, 3H), 1.92-1.82 (m, 1H), 1.56 (p, J=13.4 Hz, 3H), 1.10 (d, J=6.1 Hz, 3H). *2H under DMSO. LCMS m/z 424.23 [M+H]⁺.

Compounds 17-26 were prepared following the method described for the preparation of compound 16.

TABLE 3
Structure and Physiochemical Data for Compounds 17-26

| Compound | Aldehyde | Structure | ¹H NMR, LCMS M + 1 |
|---|---|---|---|
| 17 | 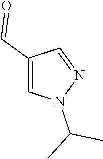 C18 | 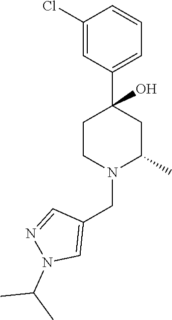 [SMILES: CC(C)n1cc(CN2CC[C@](O)(c3cccc(Cl)c3)C[C@@H]2C)cn1] | ¹H NMR (400 MHz, DMSO-d₆) δ 7.64 (s, 1H), 7.48 (t, J = 1.9 Hz, 1H), 7.38 (d, J = 7.9 Hz, 1H), 7.36-7.29 (m, 2H), 7.28-7.16 (m, 1H), 4.89 (s, 1H), 4.45 (hept, J = 6.7 Hz, 1H), 3.72 (d, J = 14.1 Hz, 1H), 3.46 (s, 1H), 2.66-2.35 (m, 2H), 1.87 (td, J = 12.8, 4.4 Hz, 1H), 1.65-1.47 (m, 3H), 1.39 (d, J = 6.6 Hz, 6H), 1.11 (d, J = 6.1 Hz, 3H). LCMS m/z 348.32 [M + H]⁺ |
| 18 | 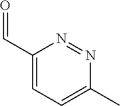 C19 | 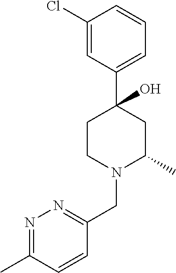 | ¹H NMR (400 MHz, DMSO-d₆) δ 7.63 (d, J = 8.5 Hz, 1H), 7.57-7.47 (m, 2H), 7.40 (d, J = 7.8 Hz, 1H), 7.33 (t, J = 7.8 Hz, 1H), 7.30-7.21 (m, 1H), 5.01 (s, 1H), 3.90 (dd, J = 232.4, 14.1 Hz, 2H), 2.81-2.70 (m, 1H), 2.59 (s, 3H), 2.66-2.50 (m, 2H), 1.86 (td, J = 12.9, 4.7 Hz, 1H), 1.69-1.46 (m, 3H), 1.10 (d, J = 6.1 Hz, 3H). LCMS m/z 332.29 [M + H]⁺ |
| 19 | 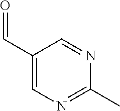 C20 | 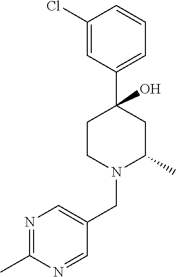 | ¹H NMR (400 MHz, DMSO-d₆) δ 8.63 (s, 2H), 7.50 (t, J = 2.0 Hz, 1H), 7.43-7.23 (m, 3H), 5.00 (s, 1H), 4.02 (d, J = 14.0 Hz, 1H), 3.25 (d, J = 13.9 Hz, 1H), 2.73-2.63 (m, 1H), 2.60 (s, 3H), 2.56-2.34 (m, 2H), 1.85 (td, J = 12.3, 5.4 Hz, 1H), 1.70- 1.44 (m, 3H), 1.12 (d, J = 6.1 Hz, 3H). LCMS m/z 332.29 [M + H]⁺ |
| 20 | 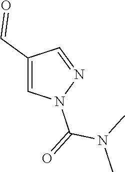 C20 | 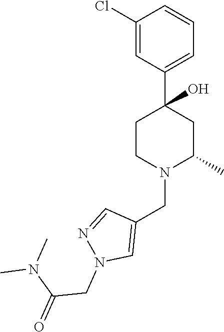 | ¹H NMR (400 MHz, DMSO-d₆) δ 7.55 (s, 1H), 7.49 (d, J = 2.0 Hz, 1H), 7.42-7.20 (m, 4H), 5.05 (s, 2H), 4.91 (s, 1H), 3.61 (dd, J = 115.5, 14.5 Hz, 3H), 3.01 (s, 3H), 2.84 (s, 3H), 2.70-2.45 (m, 2H), 1.88 (td, J = 12.8, 4.5 Hz, 1H), 1.66- 1.47 (m, 3H), 1.11 (d, J = 6.0 Hz, 3H). LCMS m/z 391.31 [M + H]⁺ |
| 21 | 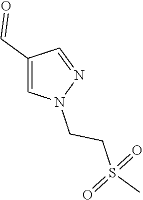 C21 | 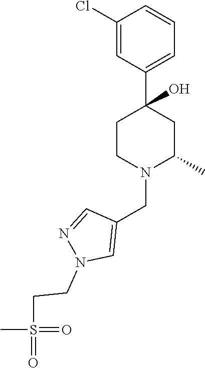 | ¹H NMR (400 MHz, DMSO-d₆) δ 7.74 (s, 1H), 7.47 (s, 1H), 7.42 (s, 1H), 7.40-7.22 (m, 3H), 4.89 (s, 1H), 4.51 (t, J = 6.8 Hz, 2H), 3.82- 3.46 (m, 5H), 2.78 (s, 3H), 2.65- 2.33 (m, 2H), 1.87 (td, J = 12.8, 4.3 Hz, 1H), 1.72-1.42 (m, 3H), 1.11 (d, J = 6.0 Hz, 3H). LCMS m/z 412.26 [M + H]⁺ |
| 22 | 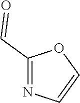 C22 | 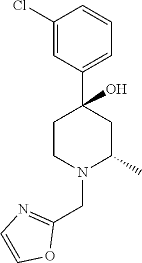 [SMILES: C[C@H]1C[C@@](O)(c2cccc(Cl)c2)CCN1Cc1ncco1] | ¹H NMR (400 MHz, DMSO-d₆) δ 8.10 (s, 1H), 7.48 (t, J = 1.9 Hz, 1H), 7.42-7.36 (m, 1H), 7.33 (t, J = 7.8 Hz, 1H), 7.29-7.23 (m, 1H), 7.19 (s, 1H), 4.93 (s, 1H), 3.88 (s, 2H), 2.70 (d, J = 34.5 Hz, 3H), 1.97-1.82 (m, 1H), 1.57 (s, 3H), 1.12 (d, J = 6.1 Hz, 3H). LCMS m/z 307.24 [M + H]⁺ |
| 23 | 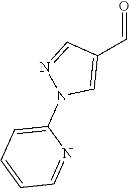 C23 | 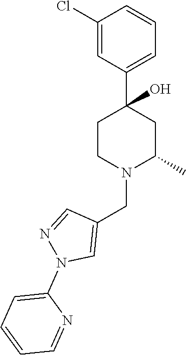 [SMILES: C[C@H]1C[C@@](O)(c2cccc(Cl)c2)CCN1Cc1cnn(-c2ccccn2)c1] | ¹H NMR (400 MHz, DMSO-d₆) δ 8.51 (s, 1H), 8.46 (dd, J = 5.0, 1.8 Hz, 1H), 7.99 (td, J = 7.8, 7.3, 1.9 Hz, 1H), 7.91 (d, J = 8.2 Hz, 1H), 7.76 (s, 1H), 7.48 (t, J = 2.0 Hz, 1H), 7.42-7.37 (m, 1H), 7.36-7.22 (m, 3H), 4.89 (s, 1H), 3.72 (dd, J = 58.6, 14.4 Hz, 2H), 2.70-2.55 (m, 3H), 1.90 (td, J = 12.6, 4.4 Hz, 1H), 1.64- 1.50 (m, 3H), 1.15 (d, J = 6.1 Hz, 3H). LCMS m/z 383.23 [M + H]⁺ |
| 24 | 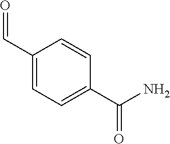 C24 | 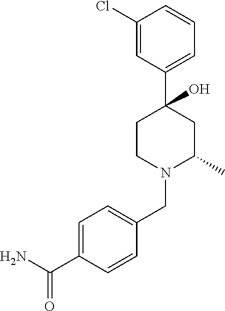 [SMILES: C[C@H]1C[C@@](O)(c2cccc(Cl)c2)CCN1Cc1ccc(C(N)=O)cc1] | ¹H NMR (400 MHz, DMSO-d₆) δ 7.93 (s, 1H), 7.82 (d, J = 8.1 Hz, 2H), 7.51 (t, J = 2.0 Hz, 1H), 7.44- 7.38 (m, 3H), 7.37-7.18 (m, 3H), 4.99 (s, 1H), 4.10 (d, J = 13.8 Hz, 1H), 3.19 (d, J = 13.8 Hz, 1H), 2.79- 2.63 (m, 1H), 2.47-2.40 (m, 1H), 1.84 (td, J = 12.7, 4.6 Hz, 1H), 1.71- 1.45 (m, 3H), 1.11 (d, J = 6.1 Hz, 3H). LCMS m/z 359.25 [M + H]⁺ |
| 25 | 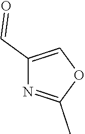 C25 | 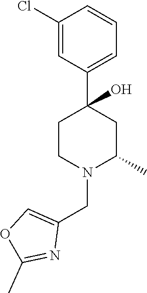 [SMILES: Cc1nc(CN2CC[C@](O)(c3cccc(Cl)c3)C[C@@H]2C)co1] | ¹H NMR (400 MHz, DMSO-d₆) δ 7.83 (s, 1H), 7.48 (t, J = 1.9 Hz, 1H), 7.42-7.23 (m, 3H), 4.91 (s, 1H), 3.72-3.46 (m, 2H), 2.70-2.58 (m, 3H), 2.38 (s, 3H), 1.93-1.82 (m, 1H), 1.55 (q, J = 12.5, 10.8 Hz, 3H), 1.11 (d, J = 6.1 Hz, 3H). LCMS m/z 321.28 [M + H]⁺ |
| 26 | 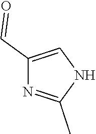 C26 | 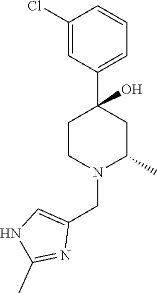 [SMILES: Cc1nc(CN2CC[C@](O)(c3cccc(Cl)c3)C[C@@H]2C)c[nH]1] | ¹H NMR (400 MHz, DMSO-d₆) δ 7.48 (t, J = 1.9 Hz, 1H), 7.44-7.31 (m, 3H), 7.26 (dt, J = 7.5, 1.8 Hz, 1H), 5.00 (d, J = 30.1 Hz, 1H), 3.86 (d, J = 13.7 Hz, 1H), 3.30 (d, J = 13.8 Hz, 1H), 2.81-2.42 (m, 3H), 2.14 (s, 3H), 1.84 (td, J = 13.5, 12.5, 7.2 Hz, 1H), 1.72-1.42 (m, 3H), 1.19 (d, J = 6.1 Hz, 3H). LCMS m/z 320.28 [M + H]⁺ |

### Example 2. Assays for Detecting and Measuring APOL1 Inhibitor Properties of Compounds

### MultiTox-Fluor Multiplex Cytotoxicity Assay

The MultiTox-Fluor Multiplex Cytotoxicity Assay is a single-reagent-addition, homogeneous, fluorescence assay that measures the number of live and dead cells simultaneously in culture wells. The assay measures cell viability and cytotoxicity by detecting two distinct protease activities. The live-cell protease activity is restricted to intact viable cells and is measured using a fluorogenic, cell-permeant peptide glycyl-phenylalanylamino fluorocoumarin (GF-AFC) substrate. The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R¹¹⁰ Substrate) is used to measure dead-cell protease that has been released from cells that have lost membrane integrity. A ratio of dead to live cells is used to normalize data.

Briefly, the tet-inducible transgenic APOL1 T-REx-HEK293 cell lines were incubated with 50 ng/mL tet to induce APOL1 in the presence of 3-(2-(4-fluorophenyl)-1H-indol-3-yl)-N-((3S,4R)-4-hydroxy-2-oxopyrrolidin-3-yl)propenamide at 10.03, 3.24, 1.13, 0.356, 0.129, 0.042, 0.129, 0.0045, 0.0015, 0.0005 μM in duplicate for 24 hours in a humidified 37° C. incubator. The MultiTox reagent was added to each well and placed back in the incubator for an additional 30 minutes. The plate was read on the EnVision plate reader. A ratio of dead to live cells was used to normalize, and data was imported, analyzed, and fit using Genedata Screener (Basel, Switzerland) software. Data was normalized using percent of control, no tet (100% viability), and 50 ng/mL tet treated (0% viability),and fit using Smart Fit. The reagents, methods, and complete protocol for the MultiTox assay are described below.

TABLE 4
Reagents Used in the Multi-Tox Assay

| Reagent | Catalog Number | Vendor |
|---|---|---|
| 384 well, transparent, flat | 356663 | Corning (Corning, |
| bottom tissue culture | | NY) |
| treated, Poly-D lysine | | |
| coated | | |
| 384 well round bottom | 3656 | CoStar (Corning, NY) |
| polypropylene plates | | |
| Universal plate lids | 250002 | Thermo Fisher |
| | | (Waltham) |
| Axygen 30 μL tips for | VT-384-31UL-R-S | Corning (Corning, |
| Bravo 384 well | | NY) |
| MultiTox-Fluor Multiplex | G9202 | Promega (Madison, |
| Cytotoxicity Assay | | WI) |
| 225 cm² flask, angled neck, | 431082 | Corning (Corning, |
| treated, vented cap | | NY) |
| Dulbecco's Phosphate- | 14190-136 | Thermo Fisher |
| Buffered Saline (DPBS), | | (Waltham) |
| calcium and magnesium- | | |
| free | | |
| Dulbecco's Modified Eagle | 11960-077 | Thermo Fisher |
| Medium (DMEM), high | | (Waltham) |
| glucose, no glutamine, no | | |
| sodium pyruvate | | |
| Fetal Bovine Serum (FBS), | 631368 | Takara (Kusatsu, |
| tetracycline-free, US- | | Japan) |
| Sourced | | |
| L-Glutamine, 200 mM | 25030-081 | Thermo Fisher |
| | | (Waltham) |
| Penicillin-Streptomycin, | 15140-122 | Thermo Fisher |
| 10,000 Units/mL | | (Waltham) |
| Blasticidin S HCl, 10 | A11139-03 | Thermo Fisher |
| mg/mL | | (Waltham) |
| Tetracycline hydrochloride | T7660 -5G | Sigma (St. Louis, MO) |
| Puromycin dihydrochloride, | A11138-03 | Thermo Fisher |
| 10 mg/mL | | (Waltham) |
| Trypsin-EDTA | 25300-054 | Thermo Fisher |
| | | (Waltham) |

TABLE 5
Equipment Used in the Multi-Tox Assay

| Instrument | Model | Supplier | Location |
|---|---|---|---|
| Bravo | 16050-101 | Agilent | Santa Clara, CA |
| | | Technologies | |
| Multidrop Combi | N/A | Thermo Scientific | Waltham, MA |
| EnVision | N/A | PerkinElmer | Waltham, MA |

### Multi-Tox Assay Protocol

Human embryonic kidney (HEK293) cell lines containing a tet-inducible expression system (T-REx™; Invitrogen, Carlsbad, CA) and Adeno-associated virus site 1 pAAVS1-Puro-APOL1 G0 or pAAVS1-Puro-APOL1 G1 or pAAVS1-Puro-APOL1 G2 Clones G0 DC2.13, G1 DC3.25, and G2 DC4.44 were grown in a T-225 flask at ˜90% confluency in cell growth media (DMEM, 10% Tet-free FBS, 2 mM L-glutamine, 100 Units/mL penicillin-streptomycin, 5 μg/mL blasticidin S HCl, 1 μg/mL puromycin dihydrochloride). Cells were washed with DPBS and then trypsinized to dissociate from the flask. Media was used to quench the trypsin, cells were then pelleted at 200 g and resuspended in fresh cell assay media (DMEM, 2% Tet-free FBS, 2 mM L-glutamine, 100 Units/mL penicillin-streptomycin). Cells were counted and diluted to 1.17×10⁶ cells/mL. 20 μL of cells (23,400/well) were dispensed in every well of a 384-well Poly-D-Lysine coated plate using the Multidrop dispenser. The plates were then incubated at room temperature for one hour.

Tetracycline is needed to induce APOL1 expression. 1 mg/mL tet stock in water was diluted to 250 ng/mL (5×) in cell assay media. 60 μL of cell assay media (no tet control) was dispensed in columns 1 and 24, and 60 μL of 5× tet in 384-PP-round bottom plate was dispensed in columns 2 to 23 with the Multidrop dispenser.

Assay ready plates from the Global Compound Archive were ordered using template 384_APOL1Cell_DR10n2_50 μM_v3. Compounds were dispensed at 200 nL in DMSO. The final top concentration was 10 μM with a 10 point 3-fold dilution in duplicate in the MultiTox assay.

20 μL was transferred from the 5× tet plate to the ARP and mixed, then 5 μL of 5× tet and the compounds were transferred to the cell plate and mixed using the Bravo. The cell plate was placed in the humidified 37° C. 5% CO₂ incubator for 24 hours.

The MultiTox-Fluor Multiplex Cytotoxicity Assay was performed in accordance with the manufacturer's protocol. After cells were incubated with tet and compound for 24 hours, 25 μL of 1× MultiTox reagent was added to each well using the Multidrop dispenser; the plates were placed on a plate shaker (600 rpm) for 2 minutes, then centrifuged briefly and placed back in the 37° C. incubator for 30 minutes. The cell viability (excitation: 400 nm, emission: 486 nm) and cytotoxicity (excitation: 485 nm, emission: 535 nm) were read using the EnVision plate reader. A ratio of dead (cytotoxicity) to live (viability) cells was reported. Data was exported and analyzed in Genedata. Data was normalized using percent of control, no tet (100% viability), and 50 ng/mL tet treated (0% viability), and fit using Smart Fit settings in Genedata.

### Potency Data for Compounds 1 to 26

The compounds of Formula I are useful as inhibitors of APOL1 activity. Table 6 below illustrates the IC₅₀ of Compounds 1 to 26 using procedures described above. The procedures above may also be used to determine the potency of any compounds of Formula I. In Table 6 below, the following meanings apply. For IP₅₀ (i.e., IC₅₀ for cell proliferation), “+++” means ≤100 nM; “++” means 100 nM to 500 nM; “+” means >500 to 5000 nM. N.D.=Not determined.

TABLE 6
Potency Data for Compounds 1 to 26

| Compound No. | IP₅₀ (nM) |
|---|---|
| 1 | ++ |
| 2 | ++ |
| 3 | + |
| 4 | +++ |
| 5 | ++ |
| 6 | + |
| 7 | +++ |
| 8 | +++ |
| 9 | +++ |
| 10 | +++ |
| 11 | ++ |
| 12 | ++ |
| 13 | +++ |
| 14 | + |
| 15 | +++ |
| 16 | ++ |
| 17 | + |
| 18 | + |
| 19 | + |
| 20 | ++ |
| 21 | +++ |
| 22 | + |
| 23 | +++ |
| 24 | ++ |
| 25 | + |
| 26 | + |

### OTHER EMBODIMENTS

This disclosure provides merely non-limiting example embodiments of the disclosed subject matter. One skilled in the art will readily recognize from the disclosure and claims, that various changes, modifications and variations can be made therein without departing from the spirit and scope of the disclosure as defined in the following claims.

1. A compound represented by the following structural formula: 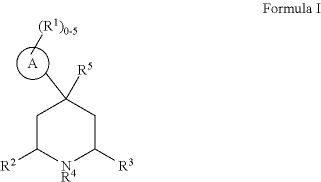
a tautomer thereof, a deuterated derivative of that compound or tautomer, or a pharmaceutically acceptable salt of any of the foregoing, wherein:
Ring A is chosen from C₆ aryl, 5- to 12-membered heterocyclyl, and 5- to 12-membered heteroaryl groups;
R¹, for each occurrence, is independently chosen from halogen, —OH, oxo, cyano, phenyl, C₁-C₆ alkyl, C₁-C₆ alkoxy, C₃-C₆ carbocyclyl, 4- to 6-membered heterocyclyl;
the 4- to 6-membered heterocyclyl of R¹ comprises one heteroatom chosen from nitrogen and oxygen;
the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
the C₁-C₆ alkoxy of R¹ is optionally substituted with 1 to 3 groups independently chosen from —OH, cyano, and halogen groups;
the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² and R³ are each independently chosen from hydrogen and C₁-C₄ alkyl groups;
R⁴ is chosen from C₁-C₆ alkyl, —C(═O)O(C₁-C₄ alkyl), and 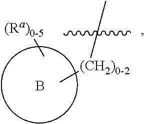
groups, wherein:
the C₁-C₆ alkyl of R⁴ is optionally substituted with 1 to 5 groups independently chosen from halogen, cyano, —OH, —NH₂, —NH(C₁-C₄ alkyl), —N(C₁-C₄ alkyl)₂,
—C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
the C₆ aryl and —O—(C₆ aryl) groups of the C₁-C₆ alkyl of R⁴ are each optionally substituted with 1 to 3 groups independently chosen from halogen and C₁-C₄ haloalkyl groups;
the Ring B of R⁴ is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
R^{a}, for each occurrence, is independently chosen from halogen, cyano, oxo, C₁-C₈ alkyl, C₁-C₆ haloalkyl, C₂-C₈ alkenyl, C₁-C₆ haloalkenyl, C₁-C₆ alkoxy,
C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —C(═O)(C₁-C₄ alkylene)OR^{k}, —C(═O)R^{k}, —C(═O)(C₁-C₄ alkylene)S(═O)_{P}R^{k}, —C(═O)(C₁-C₄ alkylene)S(═O)_{P}NR^{h}Rⁱ, —C(═O)(C₁-C₄ alkylene)NRⁱS(═O)_{P}R^{k}, —C(═O)(C₁-C₄ alkylene)NR^{h}C(═O)R^{k}, —C(═O)C(═O)R^{k}, —NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k}, —NR^{h}C(═O)NRⁱR^{j}, —NR^{h}C(═O)(C₁-C₄ alkylene)NRⁱS(═O)_{P}R^{k},
—NR^{h}S(═O)ₚR^{k}, —NR^{h}C(═O)(C₁-C₄ alkylene)S(═O)ₚR^{k}, —NR^{h}S(═O)ₚ(C₁-C₄ alkylene)C(═O)OR^{k}, —NR^{h}C(═O)[O(CH₂)_{q}]ᵣOC(═O)NR^{h}R(CH₂)_{q}[O(CH₂)_{q}]ᵣ(C₁-C₆ alkyl) (optionally substituted by 1 to 3 R^{m} groups), —NR^{h}C(═O)(C₁-C₆ alkylene)[O(CH₂)_{q}]ᵣOC(═O)NR^{h}R(CH₂)_{Q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
the C₁-C₄ alkylene in each of —C(═O)(C₁-C₄ alkylene)S(═O)ₚR^{k},
—C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)_{P}R^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)OR^{k}, —NR^{h}S(═O)ₚ(C₁-C₄ alkylene)C(═O)OR^{k}, and —NR^{h}C(═O)(C₁-C₄ alkylene)NRⁱS(═O)_{P}R^{k} of R^{a} is optionally substituted with 1 to 3 groups independently chosen from —OH,
the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k},
—OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 5- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, oxo, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, C₆ aryl (optionally substituted with 1 to 3 R^{m} groups), and 5- to 10-membered heterocyclyl groups, wherein:
R^{h}, Rⁱ, and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 3 groups independently chosen from halogen, cyano, —OH, C₁-C₄ alkoxy,
—C(═O)NH(C₁-C₄ alkyl), C₃-C₆ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups;
R^{k}, for each occurrence, is independently chosen from hydrogen, C₁-C₆ alkyl, benzyl, C₆ aryl, C₃-C₆ carbocyclyl, 5- to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), and 5- to 10-membered heteroaryl (optionally substituted with 1 to 3
—OH groups) groups; and
the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₆ alkyl, C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups, wherein:
the C₁-C₄ alkyl is optionally substituted by 1 to 3 —OH groups; and
the 5- to 10-membered heteroaryl and 5- to 10-membered heterocyclyl of any one of R^{k} are each optionally substituted with 1 to 3 groups independently chosen from halogen, cyano, —C(═O)CH₃, —NH₂, —OH, C₁-C₄ alkyl, C₁-C₄ haloalkyl, C₃-C₆ cycloalkyl, and C₁-C₄ alkoxy groups, wherein:
the C₁-C₄ alkyl is optionally substituted by 1 to 3 —OH groups;
R^{m}, for each occurrence, is independently chosen from halogen, cyano, oxo, —(CH₂)ₙC(═O)NH₂, —NH₂, —NH(C₁-C₄ alkyl), —N(C₁-C₄ alkyl)₂, C₁-C₆ alkyl, C₁-C₆ alkoxy, —C(═O)R^{k}, —S(═O)ₚR^{k}, —OR^{k}, C₃-C₆ cycloalkyl, and 5- to 10-membered heterocyclyl groups, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
R⁵ is —OH;
n is an integer chosen from 0, 1, and 2;
p, for each occurrence, is an integer independently chosen from 1 and 2; and
q and r, for each occurrence, are each an integer independently chosen from 0, 1, 2, and 3.

2. The compound, tautomer, deuterated derivative, or pharmaceutically acceptable salt according to claim 1, wherein:
Ring A is chosen from C₆ aryl and 5- to 12-membered heterocyclyl;
R¹, for each occurrence, is independently chosen from halogen, C₁-C₆ alkyl (optionally substituted with 1 to 3 groups independently chosen from halogen), and C₃-C₆ cycloalkyl groups;
R² and R³ are each independently chosen from hydrogen and C₁-C₄ alkyl groups;
R⁴ is chosen from 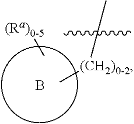
groups, wherein:
Ring B is chosen from C₃-C₁₂ cycloalkyl, 3- to 12-membered heterocyclyl, C₆ aryl, and 5- to 10-membered heteroaryl groups, wherein Ring B is optionally substituted with 1, 2, or 3 R^{a} groups; wherein:
each R^{a} is independently chosen from oxo, C₁-C₈ alkyl (optionally substituted with 1 to 3 groups selected from —C(═O)NR^{h}Rⁱ, —NR^{h}Rⁱ, and —S(═O)ₚR^{k}), C₁-C₆ haloalkyl, C₂-C₈ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl (optionally substituted with 1 to 3 oxo groups), 5- to 10-membered heteroaryl (optionally substituted with 1 to 3 groups independently chosen from halogen and C₆ aryl (optionally substituted with 1 to 3 halogen groups) groups), —C(═O)NR^{h}Rⁱ, and —C(═O)OR^{k}, wherein
R^{h}, Rⁱ, and R^{k} are as defined in claim 1; and
R⁵ is —OH.

3. The compound, tautomer, deuterated derivative, or pharmaceutically acceptable salt according to claim 1 or claim 2, wherein one of R² and R³ is hydrogen and the other is selected from from C₁-C₄ alkyl.

4. The compound, tautomer, deuterated derivative, or pharmaceutically acceptable salt according to any one of claims 1 to 3, one of R² and R³ is hydrogen and the other is methyl.

5. The compound, tautomer, deuterated derivative, or pharmaceutically acceptable salt according to claim 1 or claim 2, wherein the compound is represented by the following structural formulae IIa and IIb: 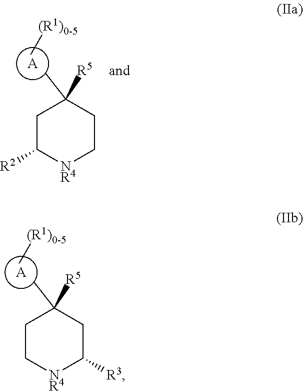
wherein R² and R³ are selected from C₁-C₄ alkyl and all variables not specifically defined herein are as defined in claim 1.

6. The compound, tautomer, deuterated derivative, or pharmaceutically acceptable salt according to claim 5, wherein of R² and R³ are methyl.

7. The compound, tautomer, deuterated derivative, or pharmaceutically acceptable salt according to any one of claims 1-6, wherein Ring A is C₆ aryl.

8. The compound, tautomer, deuterated derivative, or pharmaceutically acceptable salt according to any one of claims 1-7, wherein each Riis independently chosen from halogen and C₁-C₆ alkyl (optionally substituted with 1 to 3 halogen groups).

9. The compound, tautomer, deuterated derivative, or pharmaceutically acceptable salt according to any one of claims 1-8, wherein R⁴ is chosen from 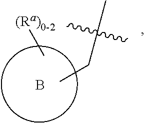
wherein Ring B and R^{a} are defined as in claim 1.

10. The compound, tautomer, deuterated derivative, or pharmaceutically acceptable salt according to claim 9, wherein Ring B is chosen from 3- to 12-membered heterocyclyl, C₆ aryl, and 5- to 10-membered heteroaryl groups, wherein Ring B is optionally substituted with 1, 2, or 3 R^{a} groups and R^{a} is as defined in claim 1.

11. The compound, tautomer, deuterated derivative, or pharmaceutically acceptable salt according to claim 10, wherein Ring B of R⁴ is chosen from: 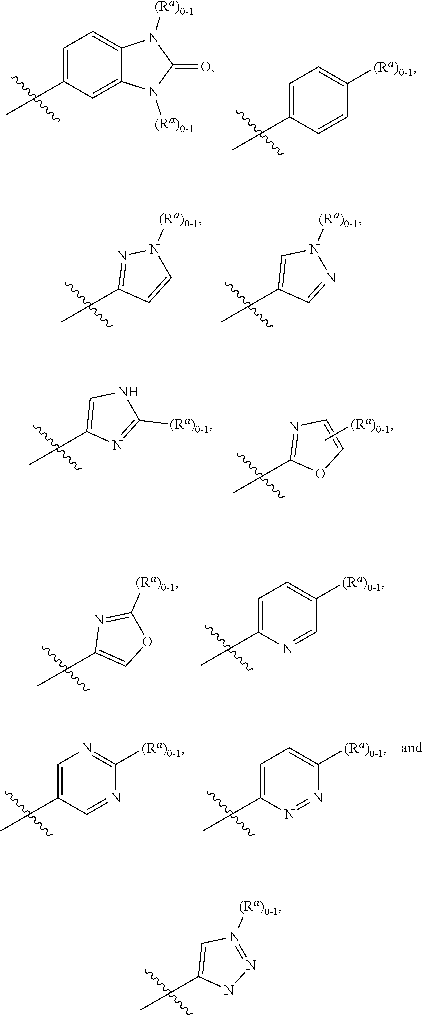
and wherein Ring B is substituted with 0 to 1 R^{a} group.

12. The compound, tautomer, deuterated derivative, or pharmaceutically acceptable salt according to claim 9 or claim 10, wherein each R^{a} is independently chosen from halogen, oxo, C₁-C₈ alkyl, C₁-C₆ haloalkyl, 3- to 12-membered heterocyclyl, 5- to 10-membered heteroaryl, and —C(═O)NR^{h}Rⁱ groups, wherein:
the C₁-C₈ alkyl and the C₁-C₆ haloalkyl of R^{a} are each optionally substituted with 1 to 3 groups independently chosen from —C(═O)NR^{h}Rⁱ,
—NR^{h}Rⁱ, and —S(═O)ₚR^{k} groups; and
the 3- to 12-membered heterocyclyl of R^{a} is optionally substituted with 1 to 3 oxo groups; wherein:
the 5- to 10-membered heteroaryl of R^{a} is optionally substituted with 1 to 3 groups independently chosen from halogen and C₆ aryl (optionally substituted with 1 to 3 halogen groups) groups, wherein:
R^{h}, Rⁱ, and R^{j}, for each occurrence, are each independently chosen from hydrogen and C₁-C₆ alkyl groups; and
R^{k}, for each occurrence, is independently chosen from C₁-C₆ alkyl groups

13. The compound, tautomer, deuterated derivative, or pharmaceutically acceptable salt according to any one of claims 1 to 12, R⁴ is 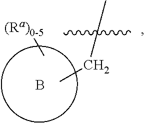
wherein Ring B is chosen from 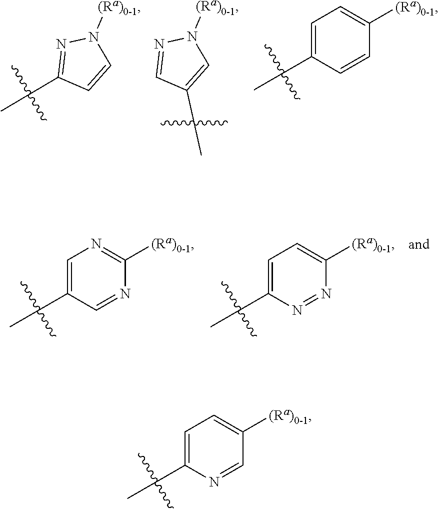
wherein R^{a} is absent, or is chosen from halogen, C₁-C₄ alkyl, and C₁-C₄ alkoxy.

14. The compound, tautomer, deuterated derivative, or pharmaceutically acceptable salt according to any one of claims 1 to 13, R⁴ is chosen from:

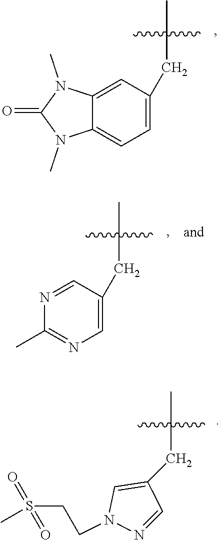

15. A pharmaceutical composition comprising a compound according to any one of claims 1 to 14.

16. A method of treating a disease mediated by ApoL1, comprising administering a compound according to any one of claims 1 to 14 or a pharmaceutical composition according to claim 15.

17. The method of treating focal segmental glomerulosclerosis (FSGS),comprising administering a compound according to any one of claims 1 to 14 or a pharmaceutical composition according to claim 15.

18. The method of treating non-diabetic kidney disease (NDKD), comprising administering a compound according to any one of claims 1 to 14 or a pharmaceutical composition according to claim 15.

19. The method of treating cancer mediated by ApoL1, comprising administering a compound according to any one of claims 1 to 14 or a pharmaceutical composition according to claim 15.

20. The method of treating cancer according to claim 19, wherein the cancer is pancreatic cancer.

21. The method of treating according to any one of claims 16 to 20, wherein the patient to be treated possesses an APOL1 genetic variants.

22. The method of treating according to claim 21, wherein the genetic variant is chosen from G1: S342G:I384M and G2: N388del:Y389del.

23. A method of inhibiting APOL1 activity comprising contacting said APOL1 with at least one compound, tautomer, deuterated derivative, or pharmaceutically acceptable salt according to any one of claims 1 to 14, or a pharmaceutical composition according to claim 15.

24. Use of a compound according to any one of claims 1 to 14 in the manufacture of a medicament for the treatment of an ApoL1 mediated disease.

25. Use of a compound according to any one of claims 1 to 14 in the manufacture of a medicament for the treatment of FSGS.

26. Use of a compound according to any one of claims 1 to 14 in the manufacture of a medicament for the treatment of NDKD.

27. Use of a compound according to any one of claims 1 to 14 in the manufacture of a medicament for the treatment of cancer mediated by ApoL1.

28. Use of a compound according to any one of claims 1 to 14 in the manufacture of a medicament for the treatment of pancreatic cancer mediated by ApoL1.

29. Use of a compound according to any one of claims 1 to 14 in the manufacture of a medicament for inhibiting the activity of ApoL1 in a patient in need thereof.

30. A compound according to any one of claims 1 to 14, or a pharmaceutical composition according to claim 15, for use in inhibiting the activity of ApoL1 in a patient in need thereof.

31. A compound according to any one of claims 1 to 14, or a pharmaceutical composition according to claim 15, for use in treating an ApoL1 mediated disorder.

32. A compound according to any one of claims 1 to 14, or a pharmaceutical composition according to claim 15, for use in treating FSGS.

33. A compound according to any one of claims 1 to 14, or a pharmaceutical composition according to claim 15, for use in treating NDKD.

34. A compound according to any one of claims 1 to 14, or a pharmaceutical composition according to claim 15, for use in treating cancer mediated by ApoL1.

35. A compound according to any one of claims 1 to 14, or a pharmaceutical composition according to claim 15, for use in treating pancreatic cancer mediated by ApoL1.
